(19) United States
(12) Patent Application Publication
(10) Pub. No.: US 2026/0000342 A1
(43) Pub. Date: Jan. 1, 2026

(54) VESTIBULAR AND OCULOMOTOR ASSESSMENT UTILIZING VIDEONYSTAGMOGRAPHY DATA AND POSTUROGRAPHY DATA

(71) Applicant: Cognuro, LLC, Orem, UT (US)

(72) Inventors: John D. Hatch, Provo, UT (US); Mike Fischer, Castle Rock, CO (US)

(73) Assignee: Cognuro, LLC, Orem, UT (US)

(21) Appl. No.: 19/251,605

(22) Filed: Jun. 26, 2025

Related U.S. Application Data
(60) Provisional application No. 63/664,497, filed on Jun. 26, 2024.

(51) Int. Cl. A61B 5/00; A61B 3/00; A61B 3/113; A61B 3/14; A61B 5/11; A61B 5/16; G16H 30/40

(52) U.S. Cl. CPC A61B 5/4023; A61B 3/0025; A61B 3/113; A61B 3/145; A61B 5/1103; A61B 5/1128; A61B 5/163; A61B 5/165; A61B 5/6803; A61B 5/702; G16H 30/40; A61B 2562/0252

(57) ABSTRACT

Assessing function of vestibular and oculomotor systems based on data output by a videonystagmography system and a force platform. A system includes a videonystagmography headset comprising an image sensor, wherein the image sensor outputs a video stream comprising a plurality of images of eyes of a user captured when the user wears the videonystagmography headset for a gaze test duration. The system includes a force platform comprising a load cell, wherein the force platform outputs a data stream indicating a plurality of tilt positions of the force platform captured when the user stands on the force platform for a balance test duration.

### CROSS-REFERENCE TO RELATED APPLICATIONS

This application claims the benefit of U.S. Provisional Application No. 63/664,497, filed Jun. 26, 2024, which is incorporated herein by reference in its entirety, including but not limited to those portions that specifically appear hereinafter, the incorporation by reference being made with the following exception: In the event that any portion of the above-referenced provisional application is inconsistent with this application, this application supersedes said above-referenced provisional application.

### TECHNICAL FIELD

The present disclosure relates to data capture and image analysis, and more particularly relates to assessing function of the vestibular system and the oculomotor system based on data output by a videonystagmography (VNG) system and force platform.

### BACKGROUND

In some cases, it is important to identify and assess abnormalities within the vestibular system and oculomotor system of a patient. All living organisms monitor their environment, and one critical aspect of that environment is gravity and the orientation of the body with respect to gravity. The vestibular system performs important tasks and engages in reflex pathway that are responsible for making compensatory movements and adjustments in body positions. The vestibular system also engages pathways that project to the cortex to provide perceptions of gravity and movement.

Further, the eyes and the oculomotor system are the only sensory system that gives input to each area of the brain. Some of those areas participate in regulating posture, movement, balance, and sensory input. The oculomotor system is composed of pathways connecting various parts of the brain dealing with controlling emotions, heart rate, breathing, sleeping, vision, personality, higher thinking, and much more.

When a person looks up, down, right, or left, and whether the person looks fast or slow, each of these movements and directions is controlled by varying parts of the person's brain. The VNG system measures the person's brain by tracking eye movements such as gaze holds, pursuits, saccades, nystagmus, and optokinetic reflexes. The test data collected helps objectively document abnormal eye movements. In some cases, abnormalities in eye function are due to lesions/breakdowns in specific areas of the brain. By identifying abnormalities in eye movement, practitioners may potentially identify dysfunctional parts of the person's brain.

In some cases, a conventional videonystagmography (VNG) system is utilized to assess abnormalities in vestibular and oculomotor systems. However, conventional VNG systems lack the precision to closely track eye movements, capture images of eye movements, and then assess the images to determine whether the eye movements are smooth, precise, and accurate.

Additionally, in some cases, posturography of a patient is assessed based on movement of a conventional force platform. However, conventional posturography methods fail to capture center of pressure data for certain combinations of assessment protocols for determining balance in various testing conditions.

What is needed are improved systems, methods, and devices for assessing health of the vestibular system and/or oculomotor system based on VNG data. Additionally, what is needed are improved systems, methods, and devices for assessing health of the vestibular system and/or oculomotor system based upon center of pressure data.

In light of the foregoing, disclosed herein are systems, methods, and devices for capturing data from a VNG system and/or a force platform. Further described herein are systems, methods, and devices for computer-executed analysis of VNG images and force measurement sensor outputs.

### BRIEF DESCRIPTION OF THE DRAWINGS

Non-limiting and non-exhaustive implementations of the present disclosure are described with reference to the following figures, wherein like reference numerals refer to like or similar parts throughout the various views unless otherwise specified. Advantages of the present disclosure will become better understood with regard to the following description and accompanying drawings where:

FIG. 1 is a schematic diagram of a system for assessing function of a vestibular system and an oculomotor system based on data output by a videonystagmography (VNG) system and a force platform;

FIG. 2 is a schematic diagram of a system and process flow for performing assessments and calculating findings based on data output by a VNG system;

FIGS. 3A and 3B are schematic flow chart diagrams of a process flow for computer-executed image analysis of data output by a VNG system;

FIG. 4 illustrates plots depicting example eye movement data calculated based on images output by a VNG system;

FIG. 5 illustrates plots depicting example eye movement data calculated based on images output by a VNG system;

FIG. 6 illustrates plots depicting example eye movement data calculated based on images output by a VNG system;

FIG. 7 illustrates plots depicting example eye movement data calculated based on images output by a VNG system;

FIG. 8 illustrates plots depicting example eye movement data calculated based on images output by a VNG system;

FIG. 9 illustrates plots depicting example eye movement data calculated based on images output by a VNG system;

FIG. 10 illustrates plots depicting example eye movement data calculated based on images output by a VNG system;

FIG. 11 illustrates plots depicting example eye movement data calculated based on images output by a VNG system;

FIG. 12 illustrates plots depicting example eye movement data calculated based on images output by a VNG system;

FIG. 13 illustrates plots depicting example eye movement data calculated based on images output by a VNG system;

FIG. 14 is a chart depicting example eye movement findings based on data output by a VNG system;

FIG. 15 is a schematic diagram of a system and process flow for performing assessments and calculating findings based on data output by a force platform;

FIG. 16 illustrates a plot depicting the position of a force platform over the course of a balance test duration wherein a user stands upon the force platform under certain assessment protocols;

FIG. 17 illustrates a plot depicting the position of a force platform over the course of a balance test duration wherein a user stands upon the force platform under certain assessment protocols;

FIG. 18 illustrates a plot depicting the position of a force platform over the course of a balance test duration wherein a user stands upon the force platform under certain assessment protocols;

FIG. 19 illustrates a plot depicting the position of a force platform over the course of a balance test duration wherein a user stands upon the force platform under certain assessment protocols;

FIG. 20 illustrates a plot depicting the position of a force platform over the course of a balance test duration wherein a user stands upon the force platform under certain assessment protocols;

FIG. 21 illustrates a plot depicting the position of a force platform over the course of a balance test duration wherein a user stands upon the force platform under certain assessment protocols;

FIG. 22 illustrates a plot depicting the position of a force platform over the course of a balance test duration wherein a user stands upon the force platform under certain assessment protocols;

FIG. 23 illustrates a plot depicting the position of a force platform over the course of a balance test duration wherein a user stands upon the force platform under certain assessment protocols;

FIG. 24 is a chart depicting outputs calculated based upon the force platform data illustrated in FIGS. 16-23;

FIG. 25 is a schematic flow chart diagram illustrating steps performed in a method for computer-executed image analysis of data output by a VNG system;

FIG. 26 is a schematic flow chart diagram illustrating steps performed in a method for computer-executed data stream analysis of data output by a force platform; and

FIG. 27 is a schematic block diagram of an example computing system according to an example embodiment of the systems and methods described herein.

### DETAILED DESCRIPTION

Disclosed herein are systems, methods, and devices for assessing health of the vestibular system and oculomotor system based upon a video stream output by a videonystagmography (VNG) device and a data stream output by force platform. Specifically described herein are testing protocols, assessment data points, and algorithms for identifying functional deficiencies relating to the vestibular system and/or the oculomotor system.

Impairments to the vestibular system can result in a range of symptoms and conditions, including vertigo (spinning sensation), dizziness, imbalance, and problems with spatial orientation. Impairments to the oculomotor system can affect the control and coordination of eye movements, leading to various visual disturbances of difficulties. In some cases, brain injuries or brain deficiencies can manifest as impairments to the vestibular system and/or the oculomotor system. These brain injuries and brain deficiencies may be identified and diagnosed by first determining whether the patient experiences a vestibular system impairment and/or an oculomotor system impairment.

Videonystagmography (VNG) is a diagnostic test used to evaluate the function of the vestibular system and assess eye movements, particularly nystagmus. Nystagmus is an involuntary rhythmic oscillation of the eyes that can occur due to various vestibular and neurological conditions. During a VNG test, a patient wears a VNG headset equipped with infrared cameras or sensors that track eye movements with high precision. The VNG headset is connected to a computer system that records and analyzes the eye movements. Conventional VNG systems lack the precision to closely track eye movements, capture images of eye movements, and then assess the images to determine whether the eye movements are smooth, precise, and accurate.

Posturography is a clinical assessment technique that measures and analyzes postural control and balance by evaluating how well a person maintains their center of gravity within their base of support. This may include static posturography that measures postural sway while a person stands still on a force platform configured to detect shifts in weight distribution. This may further include dynamic posturography that evaluates balance responses to controlled perturbations or changing sensory conditions. Posturography may be utilized to assess patients experiencing dizziness, vertigo, neurological conditions like Parkinson's disease or multiple sclerosis, and elderly patients at risk for falls. Posturography aids in identifying which sensor systems (visual, vestibular, or proprioceptive) may be contributing to balance problems. Conventional posturography systems fails to account for certain combinations of assessment protocols needed to make a complete assessment of vestibular function.

Conventional systems and methods for assessing the health of vestibular function and oculomotor function fail to include assessment of both VNG data and force platform data. The systems, methods, and devices described herein include improved VNG headset systems that output high-quality datasets that may be processed with a computer processor to assess the health of vestibular and oculomotor functions. Additionally, the systems, methods, and devices described herein include improved force platform systems that output data streams that may be assessed to determine the balance of a patient under varying assessment protocols.

Before the methods, systems, and devices of the present disclosure are described, it is to be understood that this disclosure is not limited to the configurations, process steps, and materials disclosed herein as such configurations, process steps, and materials may vary somewhat. It is also to be understood that the terminology employed herein is used for describing implementations only and is not intended to be limiting since the scope of the disclosure will be limited only by the appended claims and equivalents thereof.

In describing and claiming the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

A detailed description of systems, methods, and devices consistent with embodiments of the present disclosure is provided below. While several embodiments are described, it should be understood that this disclosure is not limited to any one embodiment, but instead encompasses numerous alternatives, modifications, and equivalents. In addition, while numerous specific details are set forth in the following description to provide a thorough understanding of the embodiments disclosed herein, some embodiments may be practiced without some or all these details. Moreover, for clarity, certain technical material that is known in the related art has not been described in detail to avoid unnecessarily obscuring the disclosure.

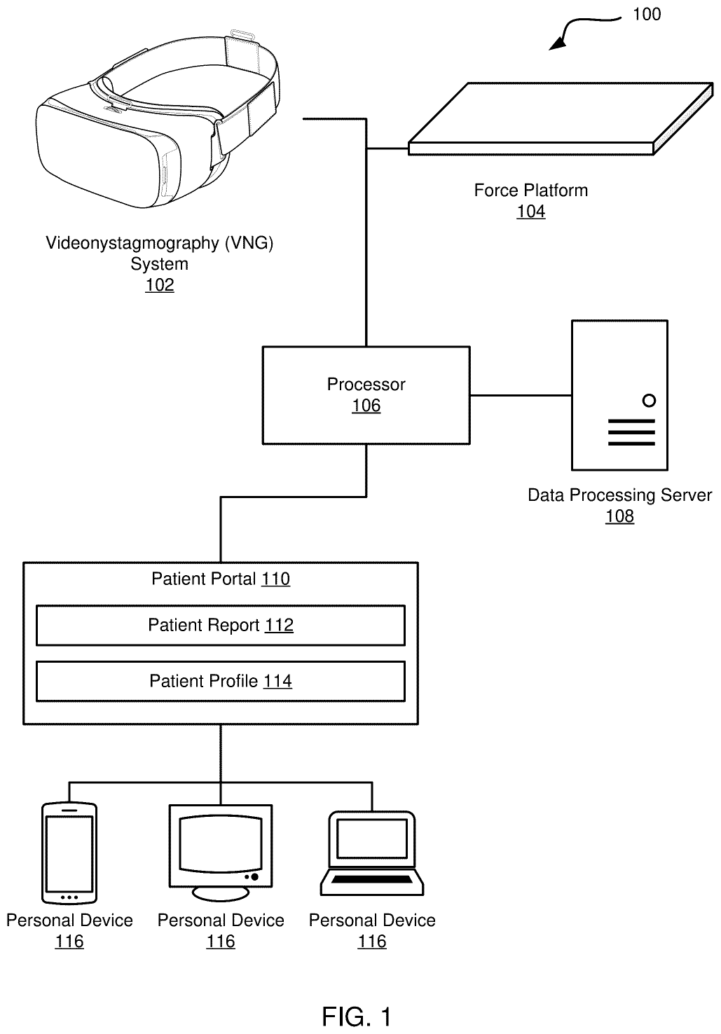

Referring now to the figures, FIG. 1 is a schematic diagram of a system 100 for assessing functional capacity of the vestibular system and/or the oculomotor system. The system 100 includes a videonystagmography (VNG) system 102 and a force platform 104. The system 100 includes one or more of a local processor 106 and/or a remote data processing server 108 that receives data output by the VNG system 102 and/or the force measurement platform 104. The processor 106 and/or data processing server 108 renders a patient portal 110 that may display patient reports 112 and/or patient profiles 114. The patient portal 110 is accessible by various personal devices 116, such as mobile phones, computers, web browsers, and so forth.

The VNG system 102 includes a VNG headset, video recording equipment, a stimulus delivery system, a monitor or display, and associated computer running applicable software. The VNG headset may include eye-tracking goggles or eye-tracking glasses. The VNG headset is typically equipped with an infrared camera or sensor to accurately track eye movements. The VNG headset is worn by the patient during the test. The video recording equipment includes one or more cameras or sensors to capture eye movements when the patient is utilizing the VNG headset. The stimulus delivery system includes one or more systems for delivering specific stimuli to the patient, such as visual stimuli or positional stimuli. The VNG system 102 may vary in design and features depending on the specific manufacturer or model.

The VNG system 102 includes a VNG headset, which is a specialized diagnostic device used to record and analyze eye movements. They VNG headset includes a high-resolution infrared camera system positioned to capture eye movements without visible light interference. The VNG headset may include a binocular recording with a separate camera for each eye. The VNG headset may typically output a video stream at a frame rate of 30-60 frames per second to capture rapid eye movements accurately.

The VNG headset may include an infrared light source to illuminate eyes for optimal pupil contrast and tracking. The light sources may be positioned around the camera lenses and operate at wavelengths that are invisible to the patient but provide excellent image contrast for eye tracking algorithms. The light sources may specifically operate at wavelengths from about 800 nm to about 900 nm, centered on 850 nm.

The VNG headset may include a lightweight, adjustable goggle frame that holds the cameras and creates a controlled visual environment. The goggle frame may include a light-tight seal to block external visual stimuli when needed. The goggle frame may include an adjustable strap to ensure secure and comfortable positioning on different head sizes.

The VNG system 102 may include an integrated or external processor 106 that executes algorithms for pupil detection and for tracking real-time eye movement analysis, calibration routines, and artifact rejection to filter out blinks and measurement errors. The processor 106 converts raw video stream data into quantifiable eye movement parameters.

The force platform 104 is designed to measure the balance of a user. The force platform 104 outputs a data stream over the course of a balance test duration, wherein a user stands on the force platform 104 under certain assessment protocol conditions. The force platform 104 tracks the position of the force platform and indicates whether the force platform has tilted forward, backward, rightward, or leftward over the course of the balance test duration.

The force platform 104 includes core sensor components, including a load cell that measures the applied force. The force platform 104 measures three-dimensional components of a single equivalent force applied to the surface of the force platform at its point of application, which is typically referred to as the center of pressure or the vertical moment of force. The force platform 104 may include a single-pedestal or multi-pedestal platform.

The force platform 104 may include an internal or external processor 106 that receives sensor data. The processor 106 may perform high-frequency sampling to capture rapid posture adjustments, while digital signal processing filters noise and extracts meaningful movement parameters. The force platform 104 may include a built-in calibration routine to establish accuracy baselines. The force platform 104 may perform static calibration protocols that account for individual sensor variations and mounting orientations.

The system 100 includes one or more of the local processor 106 or a remote data processing server 108. In many cases, the system 100 includes a local processor 106 in direct electronic communication with one or more of the VNG system 102 or the force platform 104. The local processor 106 may then provide data to the remote data processing server 108 by way of a network connection such as the Internet.

The patient portal 110 is rendered by the processor 106 and/or the data processing server 108 and is made accessible to a user by way of one or more personal devices 116. The patient portal 110 provides patient reports 112, which may include raw data, cleaned data, processed data, or charts created based on data output by the VNG system 102 and/or the force platform 104. The patient reports 112 may additionally include reports prepared by a user or administrator. The patient portal 110 additionally includes a patient profile 114, which may include information about the patient's demographics, contact information, health history, and so forth.

The patient portal 110 renders graphs and charts illustrating patient data output by the VNG system 102 and/or the force platform 104. The graphs and charts may be similar to the VNG system 102 charts illustrated herein. The graphs and charts may be similar to the force platform 104 charts illustrated herein.

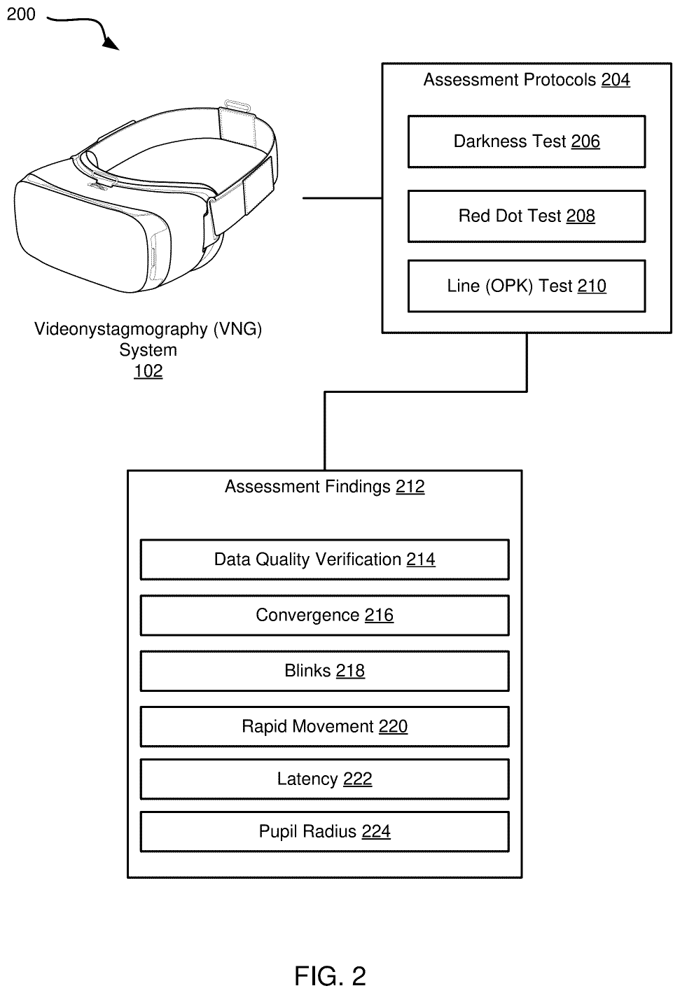

FIG. 2 is a schematic diagram of a system and process flow 200 for assessing functional capacity of the vestibular system and/or the oculomotor system. The process flow 200 begins with capturing data with a videonystagmography (VNG) system 102. The VNG system 102 outputs datasets associated with numerous assessment protocols 204, including, for example, a darkness test 206, a red dot test 208, and a line optokinetic nystagmus (OPK) test 210. The datasets are provided to a processor configured to execute one or more algorithms to determine the assessment findings 212.

Prior to calculating the assessment findings 212, the processor first ensures the quality of the data through a data quality verification 214 process. The assessment findings 212 include one or more of assessments of eye convergence 216, blinks 218, movement 220, latency 222, and pupil radius 224.

The darkness test 206 is performed when the screen of the VNG headset is turned off. Thus, the patient does not see any stimuli for the duration of the darkness test 206. The patient is told to look straight forward and keep their eyes still through the duration of the darkness test 206.

The red dot test 208 is performed with a stimuli provided to the patient through the VNG system 102. In this case, the stimuli is typically a red dot, but it should be understood that the color and formation of the stimuli could be adjusted. The patient is asked to focus on and follow movement of the red dot throughout the duration of the red dot test 208. The red dot test 208 may be performed numerous times, with the red dot moving in different directions and/or at different speeds for each iteration of the red dot test 208.

The line OPK test 210 is performed with a stimuli provided to the patient through the VNG system 102. In this case, the stimuli is typically a red line moving in front of the patient's eyes, but it should be understood that the color and formation of the stimuli could be adjusted. The patient is asked to watch the red lines as if the patient were watching television, i.e., the patient is instructed to allow their eyes to naturally watch and react to the lines.

The data quality verification 214 includes image acquisition quality checks, which may specifically include pupil visibility verification to ensure adequate infrared illumination and contrast between pupil and iris. This may include checking for proper exposure levels, wherein overexposed images may wash out pupil boundaries while underexposed images may lack sufficient detail. This may further include focus quality assessment to verify sharp pupil edges for accurate tracking algorithms. This may further include frame rate consistency monitoring to ensure no dropped frames during eye movement sequences.

The data quality verification 214 may further include pixel-to-degree calibration accuracy verification using known angular targets. The data quality verification 214 may further include lens distortion correction to ensure measurements remain accurate across the field of the view of the cameras installed within the VNG headset.

The data quality verification 214 may include signal-to-noise ratio analysis to quantify measurement precision during fixation periods. This may include drift assessment to monitor slow baseline shifts that could indicate calibration degradation. This may include saccade accuracy verification to compare detected eye movements to known calibration targets.

The convergence 216 includes analyzing the coordinated inward movement of both eyes as the eyes track objects moving closer to the face. The convergence 216 is performed based upon binocular synchronization of two cameras installed within the VNG system 102 headset. The convergence 216 assessment includes providing a controlled target movement by a visual stimulus with smooth, predictable motion that allows analysis of both voluntary convergence tracking and final convergence accuracy. Multiple approach speeds may be assessed to differentiate between slow voluntary convergence and fast fusional vergence responses.

The blinks 218 assessment includes sophisticated analysis to differentiate blinks from other eye movements to ensure the blinks do not contaminate diagnostic eye tracking data. The blinks 218 may include frame-by-frame analysis examining each video frame for sudden changes in pupil visibility or eye appearance. This may be performed with temporal smoothing to apply light filtering to reduce noise while preserving rapid blink dynamics. The blinks 218 assessment may include defining a region of interest to focus analysis on the eye area to improve computational efficiency and reduce false detections from facial movements. The blinks 218 assessment includes pupil eye tracking to monitor sudden decreases in detectable pupil area as the primary blink indicator.

The rapid movement 220 assessment includes sophisticated computer-executed analysis to accurately detect, characterize, and differentiate various types of fast eye movements. The rapid movement 220 assessment may include a velocity threshold analysis for identifying rapid eye movements (REMs). The rapid movement 220 assessment may include an acceleration analysis to examine the rate of velocity change.

The latency 222 assessment includes precise analysis of the temporal relationship between stimulus presentation and eye movement response. The latency 222 assessment includes stimulus-response synchronization, which includes temporal alignment between video frames output by the VNG system 102, and stimulus presentation provided by the VNG system 102. This may be performed with hardware synchronization using TTL triggers or frame markers to ensure precise timing correlation between stimulus onset and video acquisition. This may be performed with software timestamps to account for video processing delays and buffer times. This may include clock synchronization between stimulus delivery and camera systems to prevent timing drifting during extended recordings.

The latency 222 may measure saccadic latency indicating a delay from target appearance to saccade initiation. The latency 222 may measure smooth pursuit latency to assess delay from moving target onset to pursuit initiation. The latency 222 may measure vergence latency to examine response time for convergence/divergence movements. The latency 222 may measure vestibulo-ocular reflex latency to measure compensatory eye movement delays.

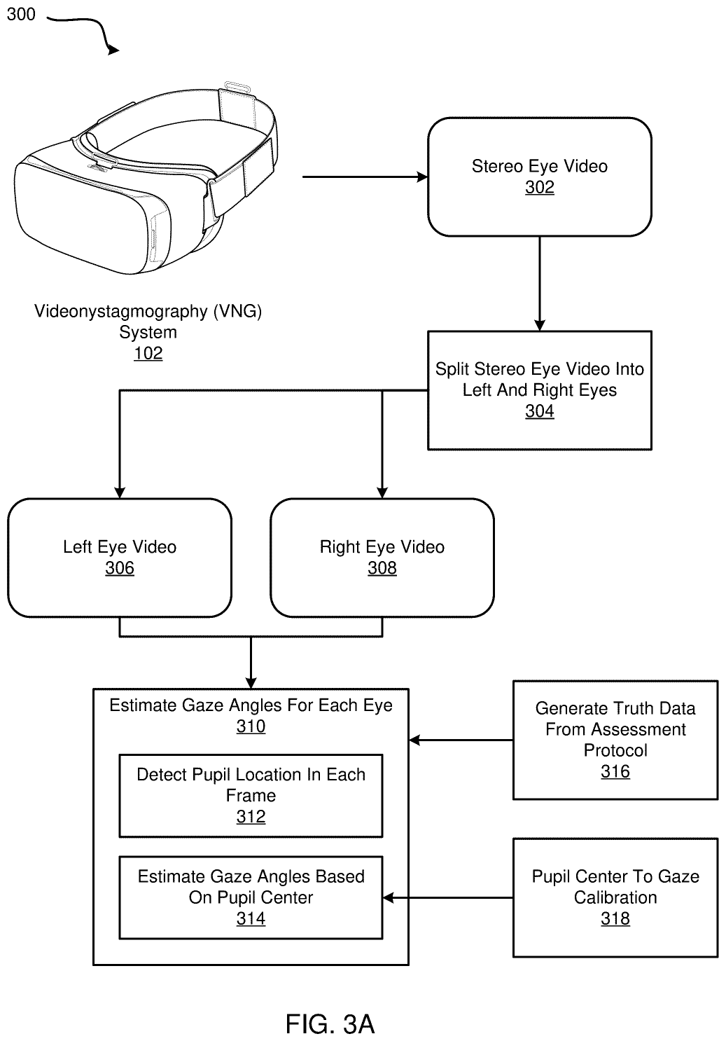

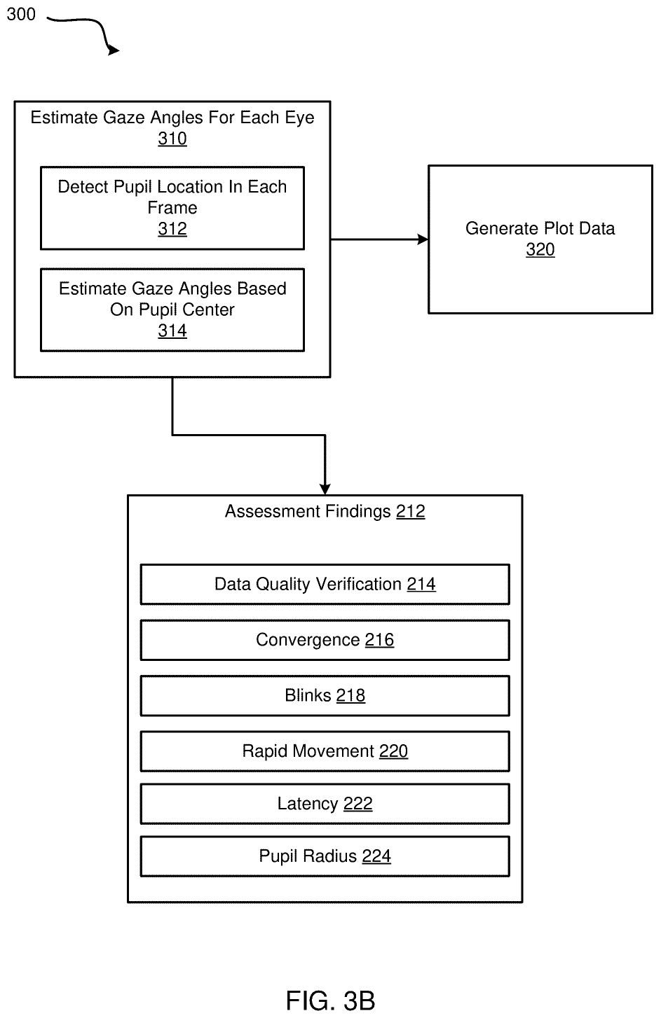

FIGS. 3A and 3B are schematic block diagrams of a process flow 300 for processing data output by the VNG system 102 and calculating the assessment findings 212. The process flow 300 begins on FIG. 3A with the VNG system 102 outputting a stereo eye video 302 to a computer or other processor.

The process flow 300 continues with splitting at 304 the stereo eye video into a left eye video 306 and a right eye video 308. The original stereo eye video 302 may include a high-definition or standard definition video provided at 30 frames per second, or an alternative and suitable frame rate. In some cases, the stereo eye video 302 is provided at a higher frame rate. This raw stereo eye video 302 is split into the separate left eye video 306 and right eye video 308. The videos 306, 308 are loaded into memory with applicable metadata including, for example, frame size, frame count, frame rate (may be converted into frames per second), and the stimuli location data for each frame.

The left eye video 306 and the right eye video 308 then separately undergo further processing. The left eye video 306 and the right eye video 308 constitute separate stacks of video frames for each eye that are independently sent down the processing pipeline. A processor executes an algorithm to estimate gaze angles for each eye 310. This algorithm includes detecting at 312 pupil location in each frame, and further includes estimating at 314 gaze angles based on the pupil center.

The process of detecting the pupil location in each frame 312 includes running each video frame (of the separate left eye video 306 and right eye video 308) through a pupil detection algorithm configured to estimate the location of the pupil and the ellipse that best fits to the pupil. The process of estimating gaze angles based on pupil center 314 is performed based on pupil center values. Based on the pupil center values, a processor normalizes the pixel values based on the estimated eye zero angle location from the truth data gaze angle values. The normalized pixel values are then passed through a pixel-to-gaze angle calibration (i.e., the pupil center to gaze calibration 318) that is derived based on numerous videos of normal eye recordings.

A processor generates at 316 truth data for the assessment protocol 204 (i.e., darkness test 206, red dot test 208, or line OPK test 210). A processor calculates the pupil center to gaze calibration at 318 based on the estimation of the gaze angles based on pupil center 314.

The process flow 300 continues on FIG. 3B with the estimated gaze angles for each eye 310 being utilized to generate plot data 320 and further being utilized to calculate the assessment findings 212.

The data quality verification 214 includes calculating a confidence value for each video frame output by the VNG system 102. The confidence value indicates how well the patient's pupil was detected within the applicable video frame. If an eye video has more than a threshold percentage of acceptable frames (i.e., frames wherein the confidence value is above a threshold), then the eye video is passed on for further processing.

The convergence 216 is defined as the eyes converging towards each other, which may alternatively be referred to as the eyes being “cross eyed.” To calculate convergence, a processor determines the value of the horizontal difference between gaze angles of the left eye and the right eye. If the value exceeds a convergence threshold, then a convergence even is counted.

The blinks 218 may be determined using the confidence value associated with the data quality verification 214. The process of counting the number of blinks may be executed by counting the occurrences of the pupil not being present in a video frame. The duration of each blink is calculated based on the number of consecutive video frames when the pupil is not present, and further based on the video frame rate. The blink threshold may be set to a minimum number of blinks and a maximum number of blinks to determine whether the patient is experiencing microsleeps during the test.

The rapid movement 220 of the eye is defined as an unexpected rapid motion that is different from what the stimuli object is doing. To calculate the occurrences of these rapid movements 220, a processor determines the upward velocity, downward velocity, leftward velocity, and rightward velocity of each eye. Those velocities are then compared to the expected velocity based on the stimuli motion. If the velocity spikes above the expected velocity, then this is counted as a rapid movement occurrence.

The latency 222 is defined as the time difference between a movement performed by the stimuli object (i.e., the stimuli provided to the patient), and the reactionary movement of the patient's eyes.

The pupil radius 224 is calculated based on data output by the VNG system 102. Part of the pupil detection process includes estimating the pupil ellipse. Using camera parameters and the estimated pupil ellipse, a processor calculates the radius of the pupil in a standard measurement unit.

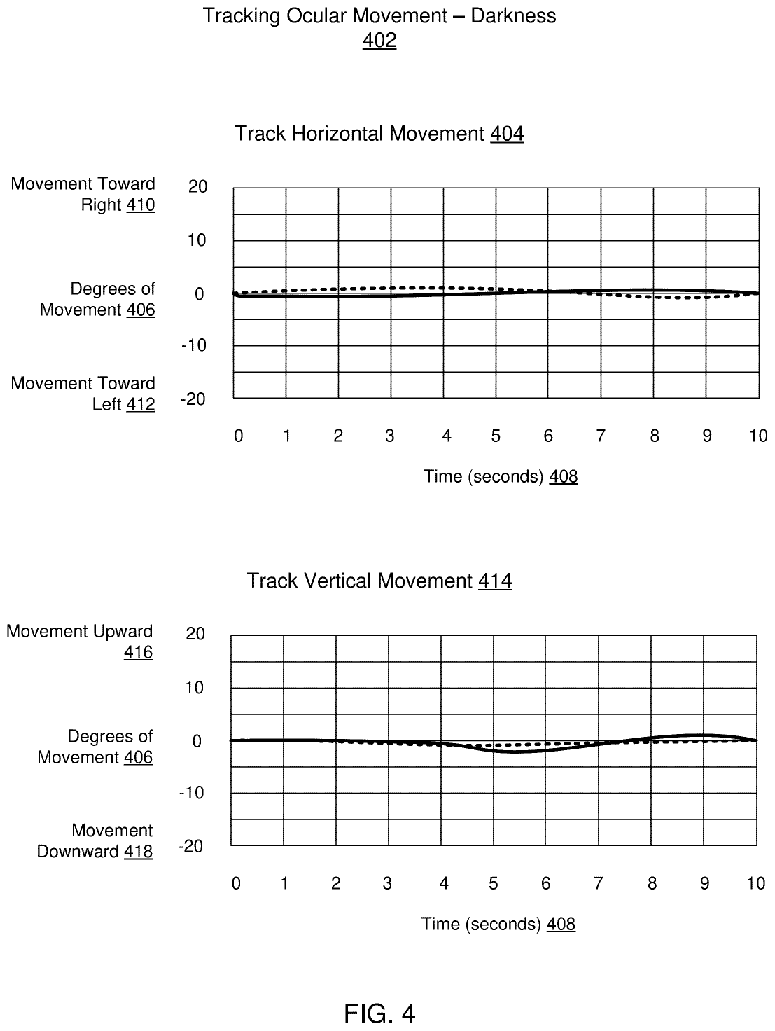

FIGS. 4-14 illustrate exemplary results of tracking ocular movement with the VNG system 102. The results may be assessed to determine assessment findings 212 as described herein, including assessing convergence 216, blinks 218, rapid movement 220, latency 222, and pupil radius 224.

FIG. 4 includes plots illustrating example results of tracking ocular movement when the VNG system 102 provides only darkness 402 to the patient, in lieu of providing a visual stimulus. FIG. 4 illustrates a plot for tracking horizontal movement 404 of the eyes of the patient and another plot for tracking vertical movement 414 of the eyes of the patient.

The horizontal movement 404 plot includes a first axis tracking the passage of time, namely, the x-axis dedicated to time 408. The horizontal movement 404 plot additionally includes a second axis tracking the movement of the eyes of the patient as measured in degrees, namely, the y-axis dedicated to degrees of movement 406. Positive increments on the degrees of movement 406 axis indicate movement toward the right 410, and negative increments on the degrees of movement 406 axis indicate movement toward the left 412.

The vertical movement 414 plot similarly includes a first axis tracking the passage of time, namely, the x-axis dedicated to time 408. The vertical movement 414 plot additionally includes a second axis tracking the movement of the eyes of the patient as measured in degrees, namely, the y-axis dedicated to degrees of movement 406. Positive increments on the degrees of movement 406 axis indicate movement upward 416, and negative increments on the degrees of movement 406 axis indicate movement downward 418.

The horizontal movement 404 and vertical movement 414 plots separately illustrate the movement of the left eye and the right of the patient. As shown in FIG. 4, the plots 404, 414 include separate lines, including a solid line and a dotted line, to indicate different measurements. One line is dedicated to illustrating the movement of the left eye of the patient, and the other line is dedicated to illustrating the movement of the right eye of the patient.

As shown in FIG. 4, when darkness 402 is provided to the patient through the VNG system 102, the eyes of the patient undergo little movement. The plots 404, 414 indicate that the patient's eyes are experiencing some movement even when the patient is instructed to avoid moving their eyes.

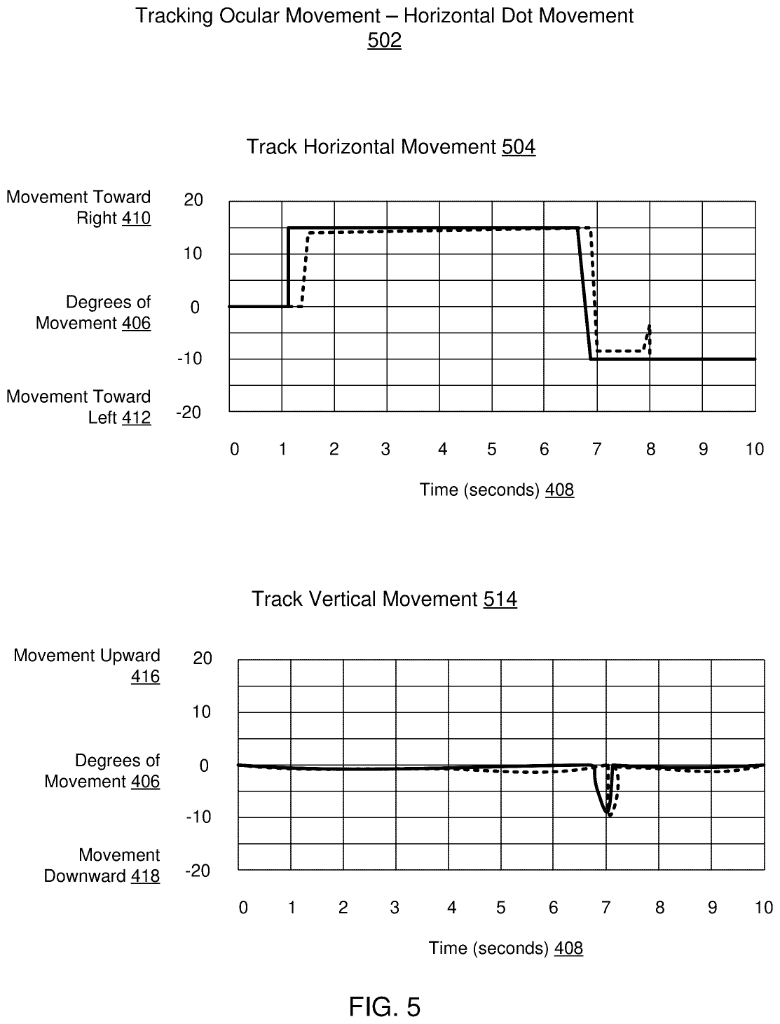

FIG. 5 includes plots illustrating example results of tracking ocular movement when the VNG system 102 provides horizontal dot movement 502 to the patient. Thus, a stimuli projector, the VNG system 102 emits a visual stimulus to the patient. This visual stimulus may be presented as a red dot that moves horizontally in front of the patient's eyes. The patient is instructed to follow the movement of the red dot. FIG. 5 illustrates a plot for tracking horizontal movement 504 of the eyes of the patient and another plot for tracking vertical movement 514 of the eyes of the patient. The axes of the plots 504, 514 are like those first illustrated in FIG. 4.

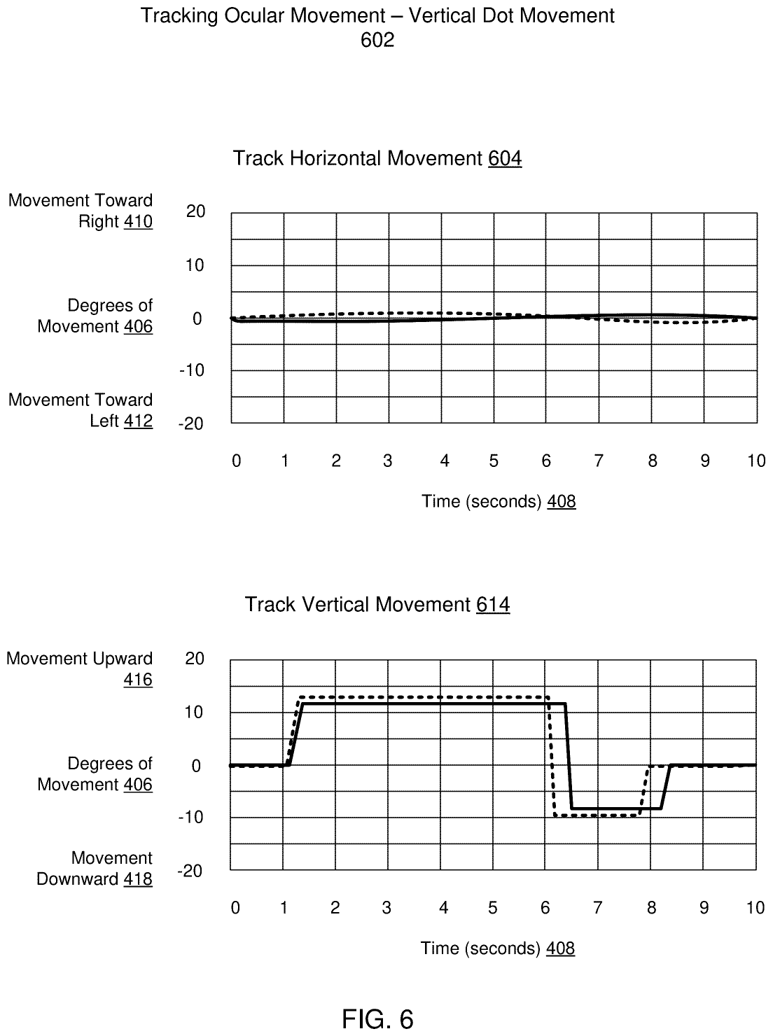

FIG. 6 includes plots illustrating example results of tracking ocular movement when the VNG system 102 provides vertical dot movement 602 to the patient. Thus, a stimuli projector of the VNG system 102 emits a visual stimulus to the patient. This visual stimulus may be presented as a red dot that moves vertically in front of the patient's eyes. The patient is instructed to follow the movement of the red dot. FIG. 6 illustrates a plot for tracking horizontal movement 604 of the eyes of the patient and another plot for tracking vertical movement 614 of the eyes of the patient. The axes of the plots 604, 614 are like those first illustrated in FIG. 4.

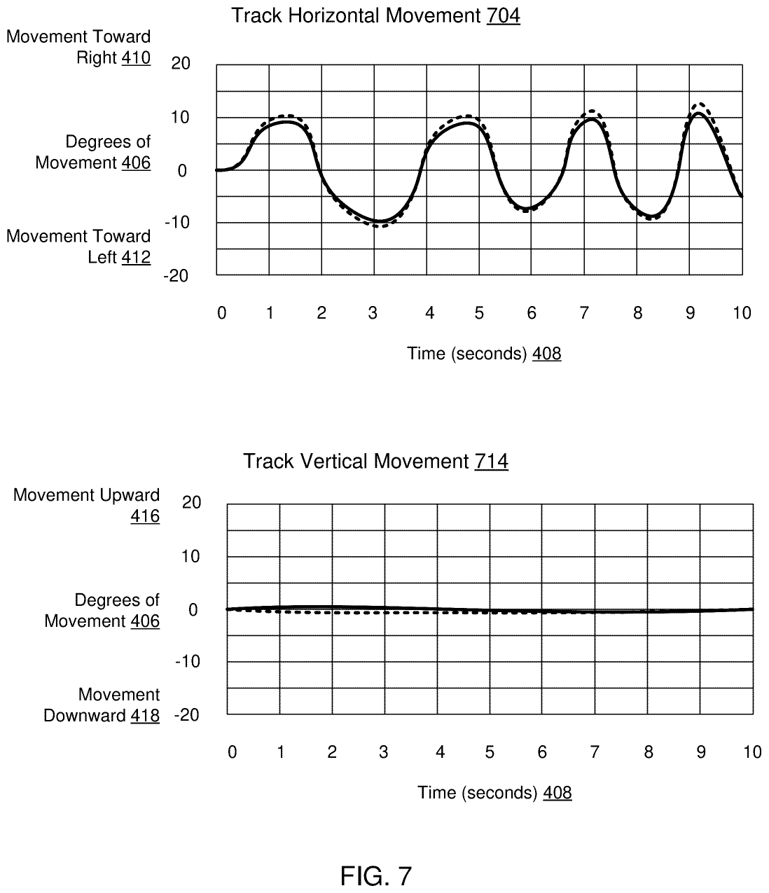

FIG. 7 includes plots illustrating example results of tracking ocular movement when the VNG system 102 provides pursuit horizontal dot movement 702 to the patient. Thus, a stimuli projector of the VNG system 102 emits a visual stimulus to the patient. This visual stimulus may be presented as a red dot that moves horizontally in front of the patient's eyes. The patient is instructed to follow the movement of the red dot. FIG. 7 illustrates a plot for tracking horizontal movement 704 of the eyes of the patient and another plot for tracking vertical movement 714 of the eyes of the patient. The axes of the plots 704, 714 are like those first illustrated in FIG. 4.

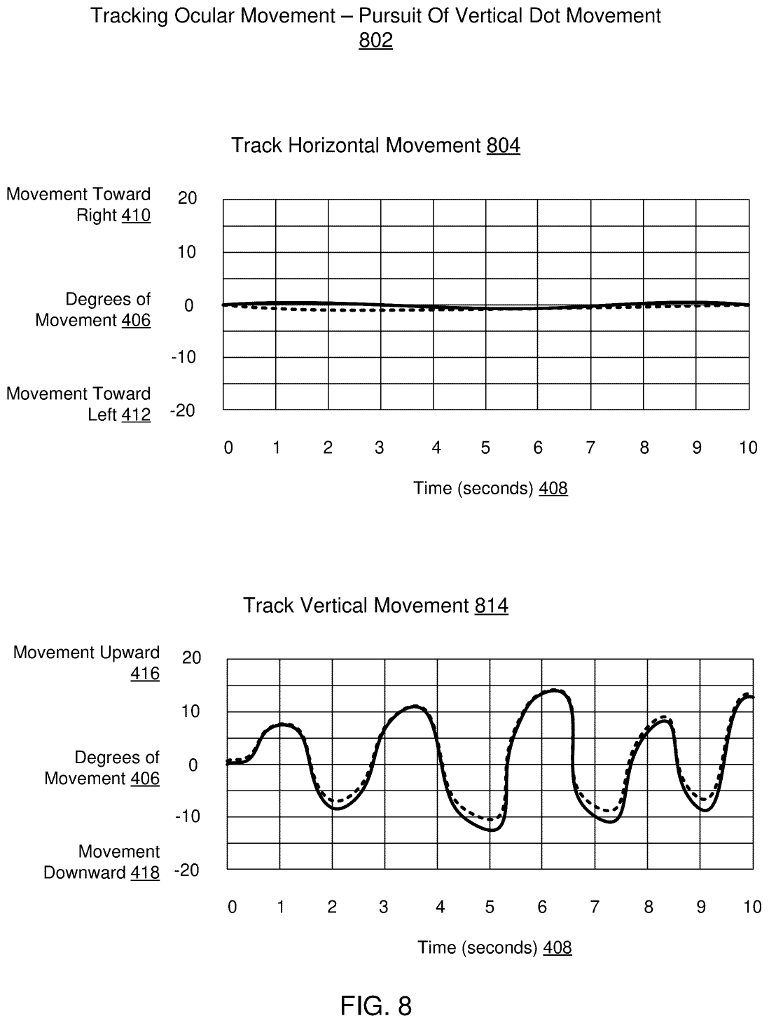

FIG. 8 includes plots illustrating example results of tracking ocular movement when the VNG system 102 provides pursuit vertical dot movement 802 to the patient. Thus, a stimuli projector of the VNG system 102 emits a visual stimulus to the patient. This visual stimulus may be presented as a red dot that moves vertically in front of the patient's eyes. The patient is instructed to follow the movement of the red dot. FIG. 8 illustrates a plot for tracking horizontal movement 804 of the eyes of the patient and another plot for tracking vertical movement 814 of the eyes of the patient. The axes of the plots 804, 814 are like those first illustrated in FIG. 4.

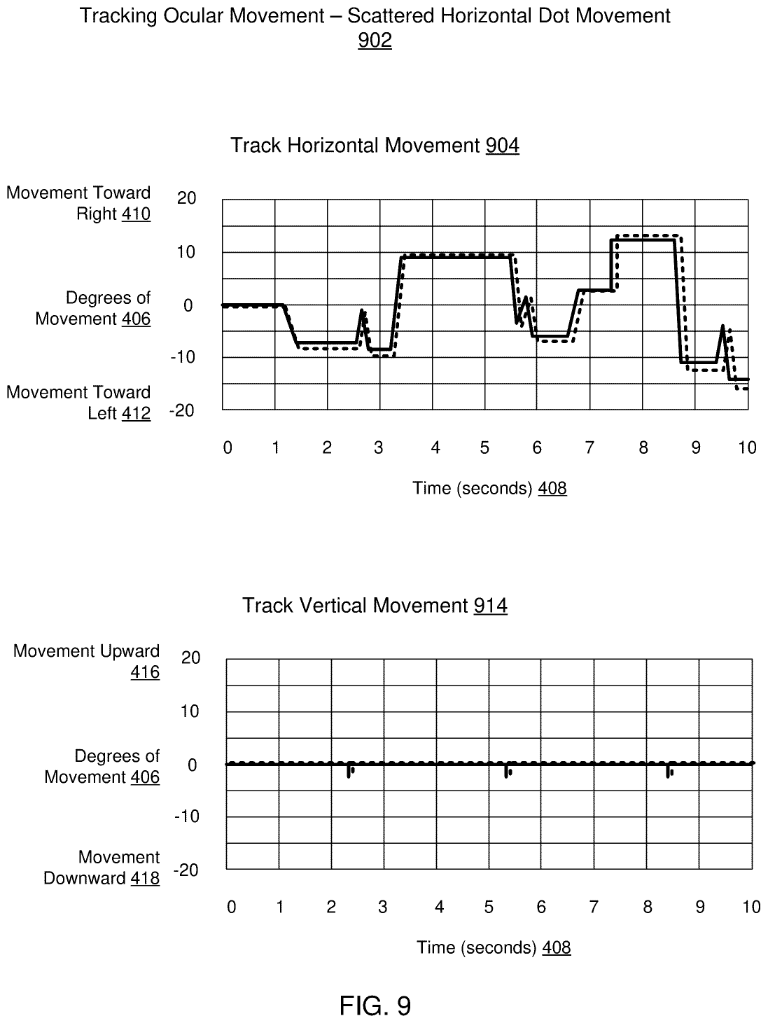

FIG. 9 includes plots illustrating example results of tracking ocular movement when the VNG system 102 provides scattered horizontal dot movement 902 to the patient. Thus, a stimuli projector of the VNG system 102 emits a visual stimulus to the patient. This visual stimulus may be presented as a red dot that moves horizontally in front of the patient's eyes. The patient is instructed to follow the movement of the red dot. FIG. 9 illustrates a plot for tracking horizontal movement 904 of the eyes of the patient and another plot for tracking vertical movement 914 of the eyes of the patient. The axes of the plots 904, 914 are like those first illustrated in FIG. 4.

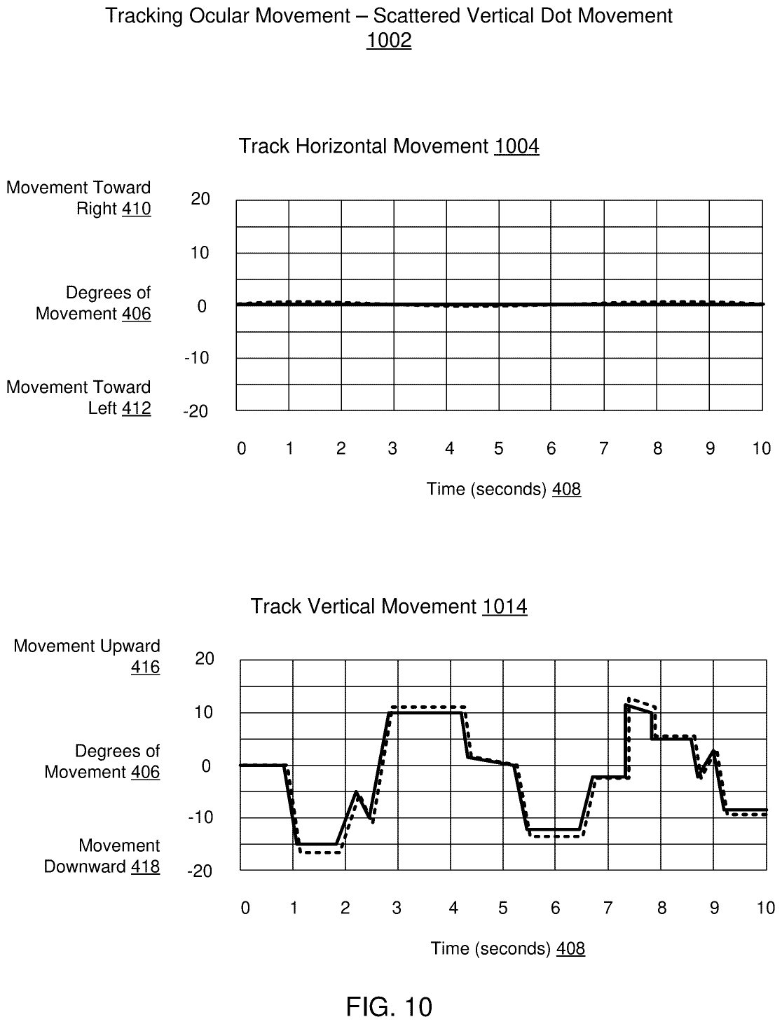

FIG. 10 includes plots illustrating example results of tracking ocular movement when the VNG system 102 provides scattered vertical dot movement 1002 to the patient. Thus, a stimuli projector of the VNG system 102 emits a visual stimulus to the patient. This visual stimulus may be presented as a red dot that moves vertically in front of the patient's eyes. The patient is instructed to follow the movement of the red dot. FIG. 10 illustrates a plot for tracking horizontal movement 1004 of the eyes of the patient and another plot for tracking vertical movement 1014 of the eyes of the patient. The axes of the plots 1004, 1014 are like those first illustrated in FIG. 4.

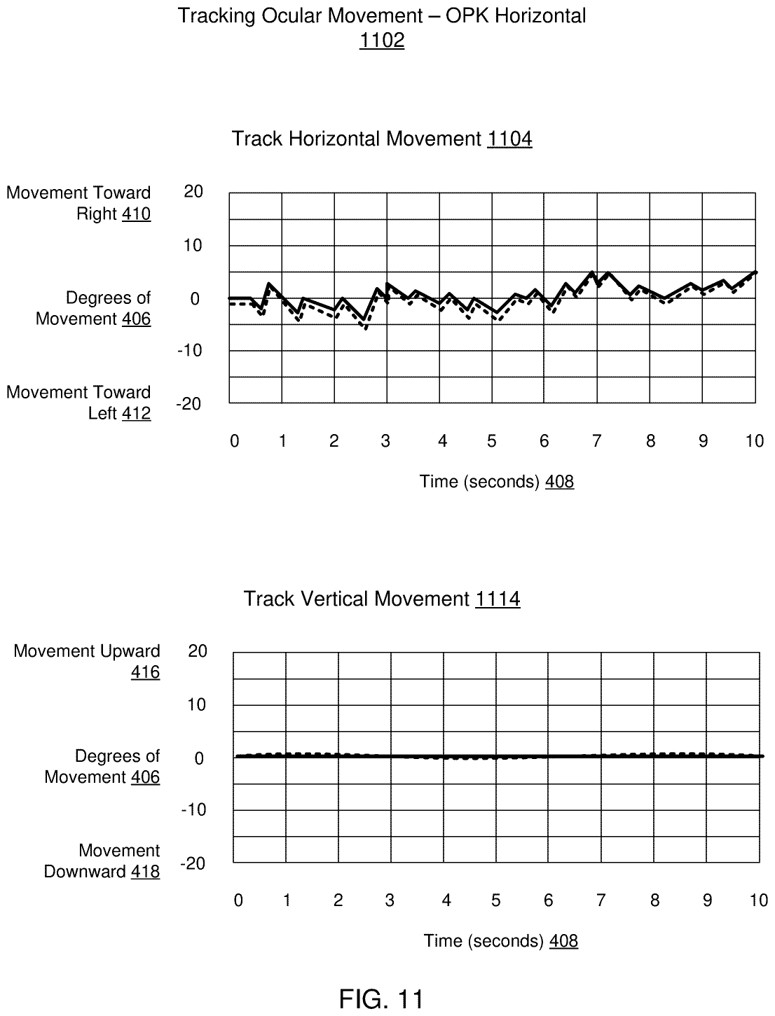

FIG. 11 includes plots illustrating example results of tracking ocular movement when the VNG system 102 provides optokinetic nystagmus (OPK) horizontal movement 1102 to the patient. Thus, a stimuli projector of the VNG system 102 emits a visual stimulus to the patient. This visual stimulus may be presented as a red line that moves horizontally in front of the patient's eyes. The patient is instructed to follow the movement of the red line. FIG. 11 illustrates a plot for tracking horizontal movement 1104 of the eyes of the patient and another plot for tracking vertical movement 1114 of the eyes of the patient. The axes of the plots 1104, 1114 are like those first illustrated in FIG. 4.

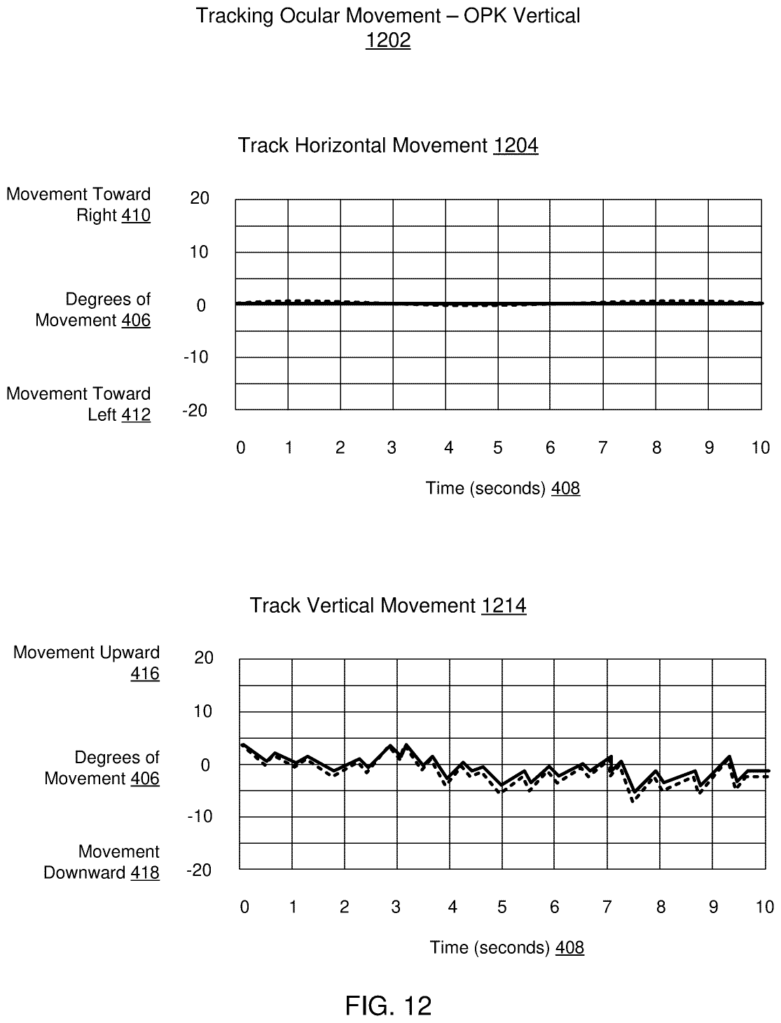

FIG. 12 includes plots illustrating example results of tracking ocular movement when the VNG system 102 provides optokinetic nystagmus (OPK) vertical movement 1202 to the patient. Thus, a stimuli projector of the VNG system 102 emits a visual stimulus to the patient. This visual stimulus may present as a red line that moves vertically in front of the patient's eyes. The patient is instructed to follow the movement of the red line. FIG. 12 illustrates a plot for tracking horizontal movement 1204 of the eyes of the patient and another plot for tracking vertical movement 1214 of the eyes of the patient. The axes of the plots 1204, 1214 are like those first illustrated in FIG. 4.

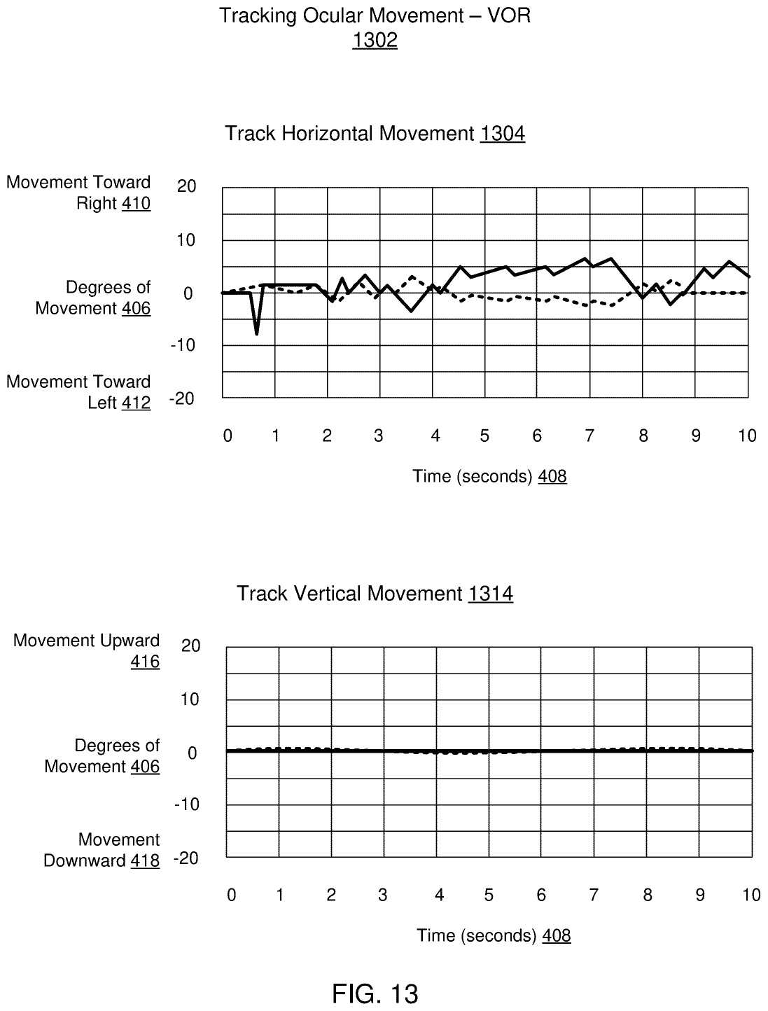

FIG. 13 includes plots illustrating example results of tracking ocular movement when the VNG system 102 provides vestibula-ocular reflex (VOR) stimuli 1302 to the patient. Thus, a stimuli projector of the VNG system 102 emits a visual stimulus to the patient. This visual stimulus may be presented as a red dot that moves horizontally in front of the patient's eyes. The patient is instructed to follow the movement of the red dot. FIG. 13 illustrates a plot for tracking horizontal movement 1304 of the eyes of the patient and another plot for tracking vertical movement 1314 of the eyes of the patient. The axes of the plots 1304, 1314 are like those first illustrated in FIG. 4.

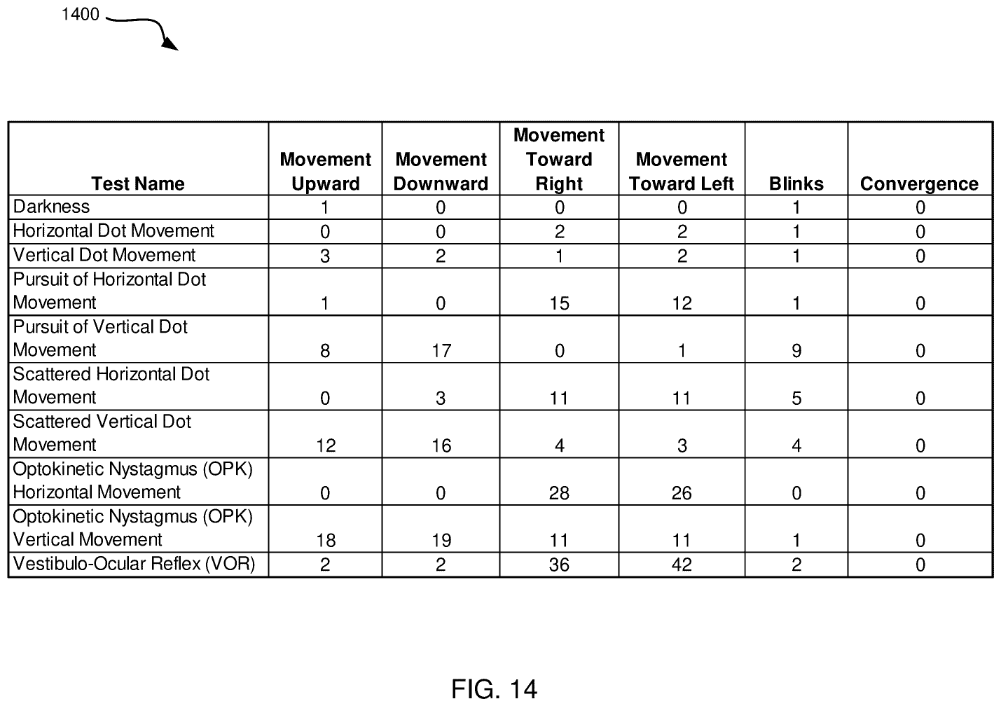

FIG. 14 is a chart illustrating example results 1400 of the tests illustrated in FIGS. 4-13. The example results 1400 may be rendered on a user interface and provided to a patient.

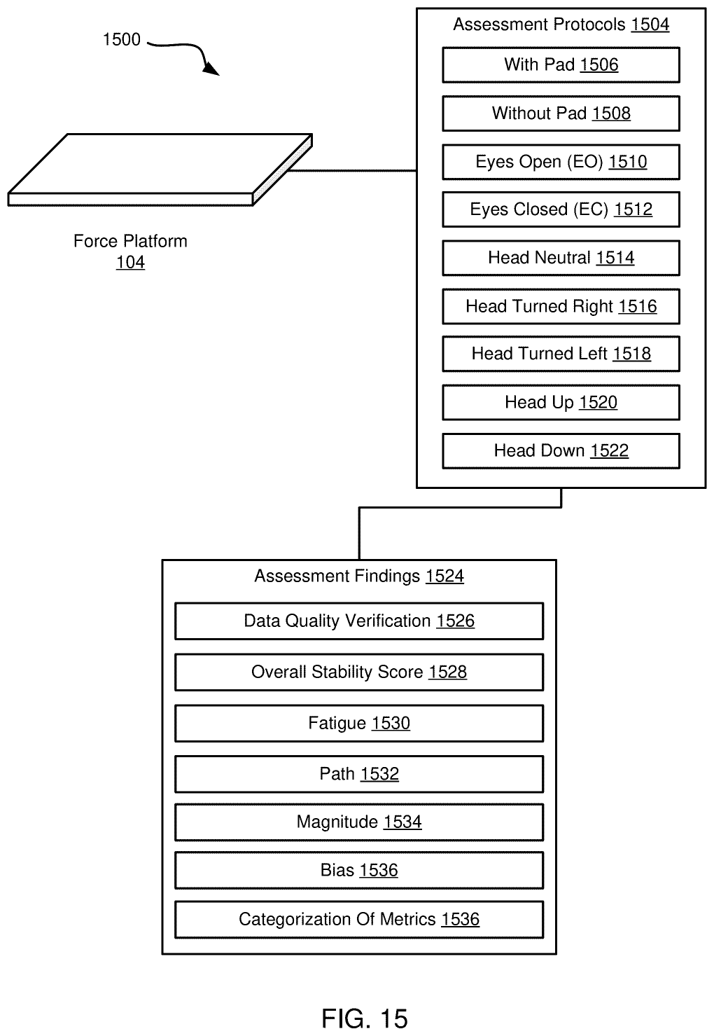

FIG. 15 is a schematic diagram of a system and process flow 1500 for assessing functional capacity of the vestibular system and/or the oculomotor system. The process flow 1500 begins with capturing data with a force platform 104. The force platform 104 is utilized as a posturography assessment aid. Posturography is a diagnostic test utilized to assess balance and postural stability. The force platform 104 measures how well a person can maintain their balance under various conditions by analyzing body movements in response to controlled disturbances and quiet standing.

The process flow 1500 is implemented to assess several aspects of balance, including sensory integration, motor control, and coordination and stability. The assessment of sensory integration includes assessing how well the sensory system, including vision, vestibular systems, and somatosensory systems, work together to maintain balance. The assessment of motor control includes assessing how muscles and joints respond to balance challenges. The assessment of coordination and stability includes assessing sway patterns and postural adjustments to reveal issues with coordination and equilibrium.

Prior to calculating the assessment findings 1524, the processor first ensures the quality of the data through a data quality verification 1526 process. The assessment findings 212 include an overall stability score 1528 and may additionally include scores relating to fatigue 1530, path 1532, magnitude 1534, and bias 1536. The assessment findings 1524 may additionally include a categorization of metrics 1538 classifying the patient as stable, weak, or unstable for each of overall stability, fatigue, path, magnitude, and bias.

The force platform 104 may be utilized to output numerous datasets based on various assessment protocols 1504. A patient may be evaluated under various combinations of assessment protocols 1504 as deemed necessary or prudent based on the patient's health history. The assessment protocols 1504 include assessing balance on the force platform 104 with a pad 1506 or without a pad 1508; assessing balance on the force platform 104 with eyes open (EO) 1510 or with eyes closed (EC) 1512; assessing balance on the force platform 104 with the head in a neutral position 1514, with the head turned to the right 1516, with the head turned to the left 1518, with the head pointed upward 1520, or with the head pointed downward 1522.

The force platform 104 outputs a plurality of datasets for a plurality of assessment protocols 1504. Each of these datasets is provided to the processor 106 (see FIG. 1) and/or the data processing server 108 (see FIG. 1) for further assessment. The findings may be rendered in the patient portal 110 (see FIG. 1) to be accessed by the patient or other healthcare providers.

In an exemplary implementation, the force platform 104 outputs a unique dataset for one or more of the following combinations of assessment protocols 1504: without pad, eyes open, and head neutral; without pay, eyes closed, and head neutral; with pad, eyes open, and head neutral; with pad, eyes closed, and head neutral; with pad, eyes closed, and head turned right; with pad, eyes open, and head turned left; with pad, eyes closed, and head turned down; or with pad, eyes closed, and head turned up.

The assessment findings 1524 include data quality verification 1526. The data quality verification 1526 is a systematic process to ensure the data meets standards for accuracy, completeness, consistency, and reliability. This may be performed by first establishing data quality dimensions for accuracy (i.e., data reflects reality), completeness (i.e., no missing critical values), consistency (i.e., uniform data formats and values), validity (i.e., data conforms to defined rules), and uniqueness (i.e., no unwanted duplicates). The data quality verification 1526 may be an automated process performed prior to performing further assessments on the data.

The assessment findings 1524 include calculating an overall stability score 1528. This includes calculating Center of Pressure (center of pressure) from each of the force measurement sensors and their relative locations to generate a calculated location for the center of the pressure applied to the force platform. The overall stability score 1528 is calculated based upon a center of pressure movement, indicating a sway path length as determined based upon force sensor data over time. This includes measuring the area of confidence ellipse containing the center of pressure movement points, and further includes tracking maximum displacement from center position. The overall stability score 1528 is further calculated based upon sway velocity and acceleration in medial-lateral and anterior-posterior directions, and further based upon peak sway velocities as indicators of balance corrections. The overall stability score 1528 may include a percentage score, such as 90-100% indicating excellent stability, 80-89% indicating good stability, 70-79% indicating fair stability, and below 70% indicating poor stability requiring attention.

The assessment findings 1524 include calculating fatigue 1530. The stability fatigue 1530 tracks how balance performance degrades over time. Stability fatigue generally manifests as progressive deterioration in postural control, which typically shows increased sway amplitude and velocity, reduced balance recovery efficiency, changed frequency characteristics of postural adjustments, and decreased ability to maintain stable positions.

The fatigue 1530 is comparison of overall movement between an initial time duration on the force platform 104 versus a final time duration on the force platform 104. Fatigue is calculated using the center of pressure values over time which gives a measure of the body's movement. The fatigue 1530 is calculated using the path length of the center of pressure during the half of the test on the force platform 104 versus the final half of the test on the force platform 104, or an alternative time duration as deemed appropriate. The fatigue 1530 is further calculated based upon the progressive sway increased, which is performed through tracking RMS sway amplitude over time and measuring path length increase per time unit. The fatigue 1530 is further calculated based upon velocity and acceleration changes, wherein peak velocities indicate compensatory movements and higher RMS acceleration suggests less efficient control. The fatigue 1530 is further calculated based upon recovery time analysis, which includes an analysis of time to return to baseline after perturbations, an amplitude of overshoot during recovery, and a number of oscillations before stabilization. The final fatigue value may be a weighted combination of the path length, RMS sway amplitude, velocity and acceleration changes, and recovery time.

The assessment findings 1524 include calculating path 1532. The path is calculated by using the time series signal representing center of pressure (center of pressure) displacement along at least one axis selected from the group consisting of a medial-lateral axis, an anterior-posterior axis, and a combined two-dimensional axis; computing a first-order temporal difference of the signal data to generate a set of incremental displacements between successive time points; determining a sway length value as the cumulative sum of the magnitudes of said incremental displacements. The path 1532 indicates the overall center of pressure distance measured by the force platform 104 during the test.

The assessment findings 1524 include calculating magnitude 1534. The magnitude 1534 indicates the how large movements were from forward to back, and from left to right. The magnitude 1534 assessment involves analyzing the amplitude and intensity of postural movements to quantify the overall scale of body sway and balance corrections. The magnitude is calculated from the center-of-pressure (center of pressure) signal data generated by the force platform, the center of pressure data including at least one of (i) a medial-lateral component and (ii) an anterior-posterior component; selecting an analysis axis from a set consisting of the medial-lateral axis and the anterior-posterior axis. For the selected analysis axis, retrieving a percentile-based displacement value, herein designated P-95, which represents a displacement magnitude exceeded by no more than five percent of the center of pressure samples acquired during the balance test. In certain embodiments the P-95 value is pre-computed as the ninety-fifth percentile of the absolute center of pressure displacements along the selected axis. Forming a sway-magnitude value by taking an absolute value of the P-95 displacement to ensure a non-negative representation of sway amplitude, thereby yielding an axis-specific sway-magnitude parameter indicative of the extreme sway envelope encompassing approximately ninety-five percent of the subject's postural excursions.

The magnitude 1534 assessment may include peak-to-peak amplitude analysis, where the maximum range of movement in each direction is calculated by finding the difference between the highest and lowest displacement values during the measurement period. This metric may be valuable for identifying boundaries of stability and understanding how far the center of mass travels during balance maintenance.

The assessment findings 1524 include calculating bias 1536. The bias 1536 indicates a direction of bias in the movements measured in the center of pressure during the test on force platform 104, including from forward to back, and/or from left to right. The bias is calculated by receiving center of pressure signal data captured from a force platform, the signal data including a plurality of time-indexed samples along at least one axis selected from (i) a medial-lateral axis and (ii) an anterior-posterior axis; and selecting an analysis axis from said at least one axis set. Then computing a bias value for the selected axis by forming a weighted arithmetic mean of the center of pressure displacements using the weighting vector w, the bias value characterizing a directional drift or offset in postural sway that progressively emerges during the test. The weight vector is proportional to a squared, normalized time value of its corresponding sample.

The assessment findings 1524 include calculating a categorization of metrics 1538, and this may be performed to categorize the stability of the patient with respect to the fatigue, path, magnitude, and bias. The categorization may include categorizing each category as “stable” (i.e., healthy, and/or normal), “weak” (i.e., slightly outside a predetermined threshold range), or “unstable” (i.e., very outside a predetermined threshold range).

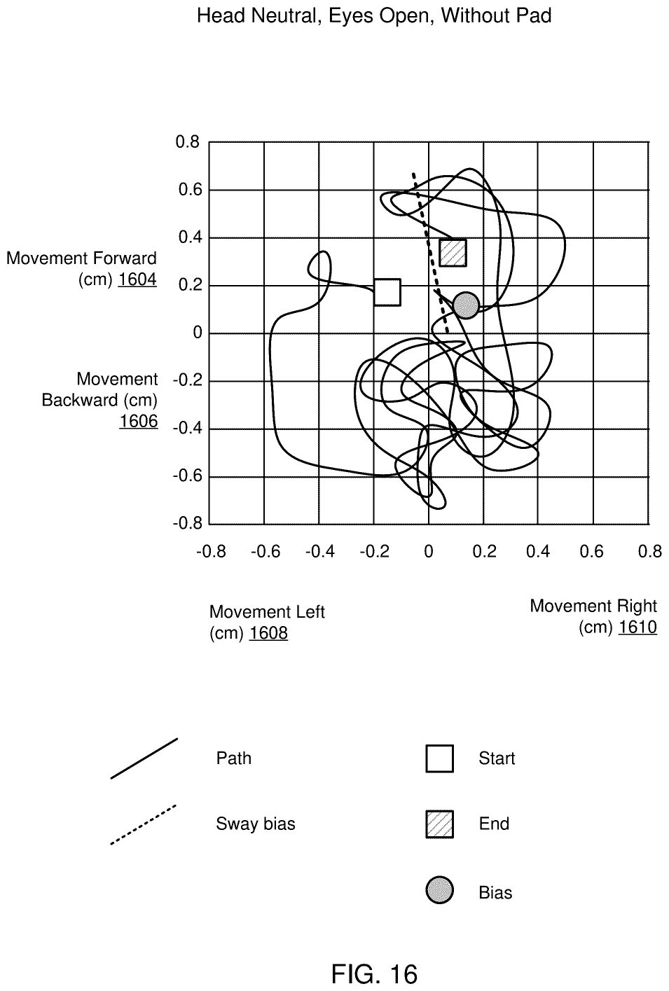

FIGS. 16-24 illustrate exemplary results of tracking posturography with a force platform 104. For all plots illustrated in FIGS. 16-23, the plot indicates the movement of the force platform 104 in terms of centimeters (cm) of movement. This is illustrated on graphs indicating movement forward 1604 along the positive y-axis, movement backward 1606 along the negative y-axis, movement to the left 1608 along the negative x-axis, and movement to the right 1610 along the positive x-axis. The solid lines indicate the position of the force platform 104 over time, with the time beginning at the starting point (see the white block), and the time ending at the ending point (see diagonal striped block). The dotted lines indicate the movement bias over the duration of the test. The shaded circle indicates the center of movement bias over the duration of the test.

FIG. 16 is a plot of exemplary results of tracking posturography with a force platform 104 under the following assessment protocols: head turned in a neutral position, eyes open, and without pad. The white block indicates the position of the force platform 104 in terms of forward/backward tilt and further in terms of rightward/leftward tilt, the dark line indicates the position of the force platform 104 in terms of forward/backward tilt and further in terms of rightward/leftward tilt over time, and the diagonal lined block indicates the final position of the force platform 104 in terms of forward/backward tilt and further in terms of rightward/leftward tilt.

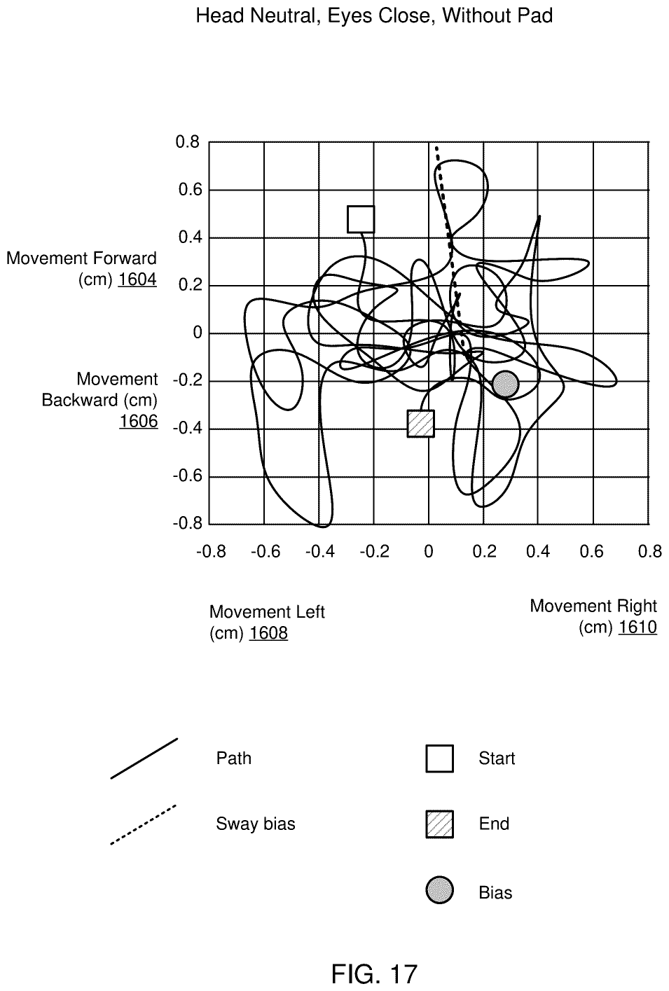

FIG. 17 is a plot of exemplary results of tracking posturography with a force platform 104 under the following assessment protocols: head turned in a neutral position, eyes closed, and without pad. The white block indicates the position of the force platform 104 in terms of forward/backward tilt and further in terms of rightward/leftward tilt, the dark line indicates the position of the force platform 104 in terms of forward/backward tilt and further in terms of rightward/leftward tilt over time, and the diagonal lined block indicates the final position of the force platform 104 in terms of forward/backward tilt and further in terms of rightward/leftward tilt.

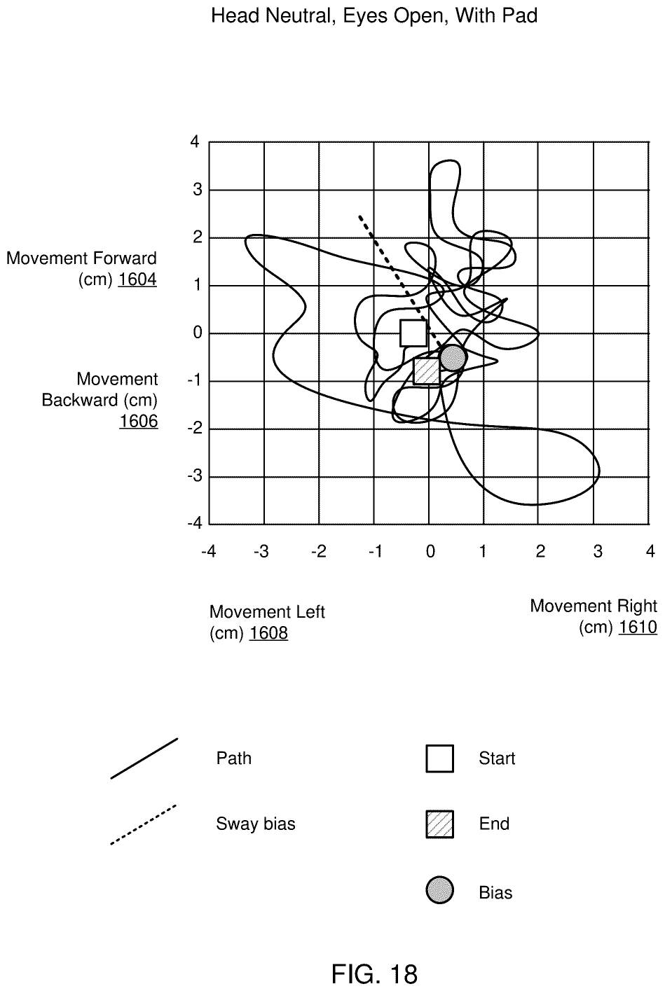

FIG. 18 is a plot of exemplary results of tracking posturography with a force platform 104 under the following assessment protocols: head turned in a neutral position, eyes open, and with pad. The white block indicates the position of the force platform 104 in terms of forward/backward tilt and further in terms of rightward/leftward tilt, the dark line indicates the position of the force platform 104 in terms of forward/backward tilt and further in terms of rightward/leftward tilt over time, and the diagonal lined block indicates the final position of the force platform 104 in terms of forward/backward tilt and further in terms of rightward/leftward tilt.

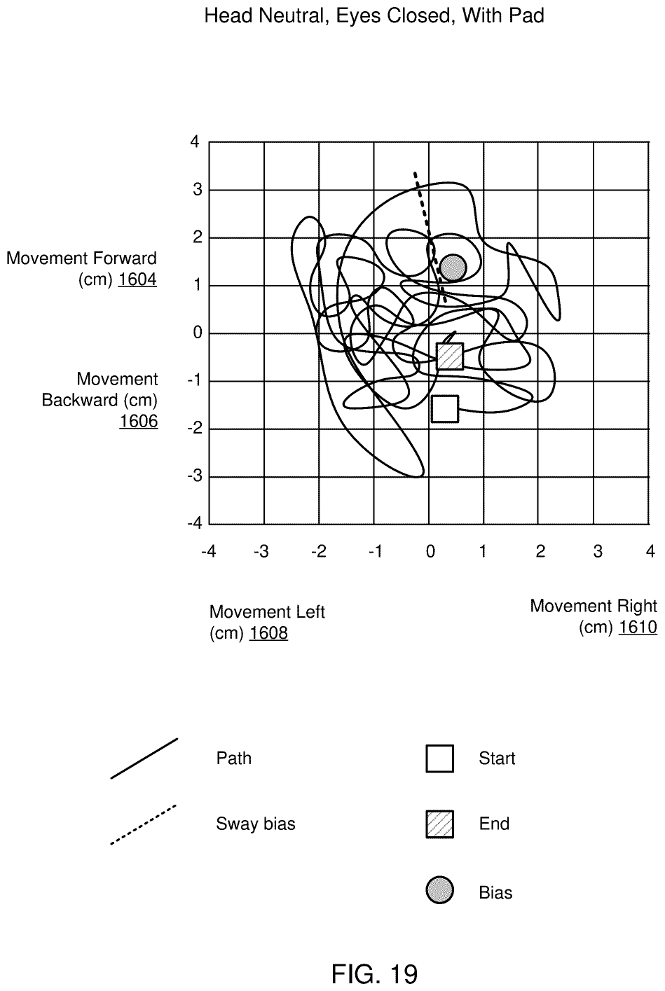

FIG. 19 is a plot of exemplary results of tracking posturography with a force platform 104 under the following assessment protocols: head turned in a neutral position, eyes closed, and with pad. The white block indicates the position of the force platform 104 in terms of forward/backward tilt and further in terms of rightward/leftward tilt, the dark line indicates the position of the force platform 104 in terms of forward/backward tilt and further in terms of rightward/leftward tilt over time, and the diagonal lined block indicates the final position of the force platform 104 in terms of forward/backward tilt and further in terms of rightward/leftward tilt.

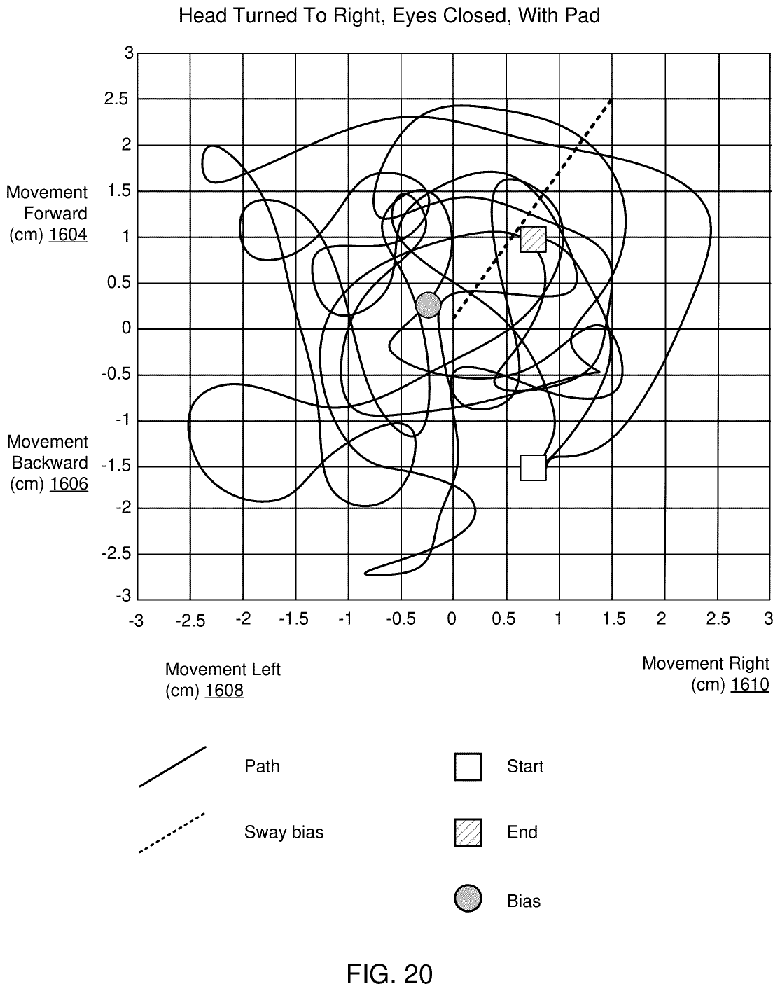

FIG. 20 is a plot of exemplary results of tracking posturography with a force platform 104 under the following assessment protocols: head turned to the right, eyes closed, and with pad. The white block indicates the position of the force platform 104 in terms of forward/backward tilt and further in terms of rightward/leftward tilt, the dark line indicates the position of the force platform 104 in terms of forward/backward tilt and further in terms of rightward/leftward tilt over time, and the diagonal lined block indicates the final position of the force platform 104 in terms of forward/backward tilt and further in terms of rightward/leftward tilt.

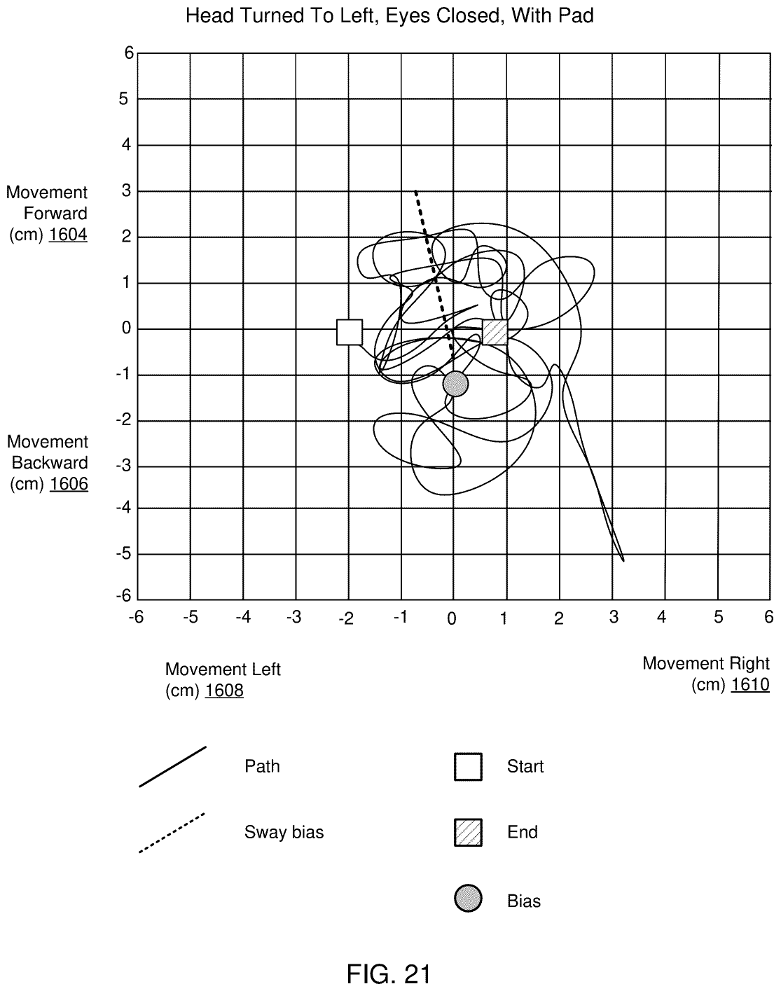

FIG. 21 is a plot of exemplary results of tracking posturography with a force platform 104 under the following assessment protocols: head turned to the left, eyes closed, and with pad. The white block indicates the position of the force platform 104 in terms of forward/backward tilt and further in terms of rightward/leftward tilt, the dark line indicates the position of the force platform 104 in terms of forward/backward tilt and further in terms of rightward/leftward tilt over time, and the diagonal lined block indicates the final position of the force platform 104 in terms of forward/backward tilt and further in terms of rightward/leftward tilt.

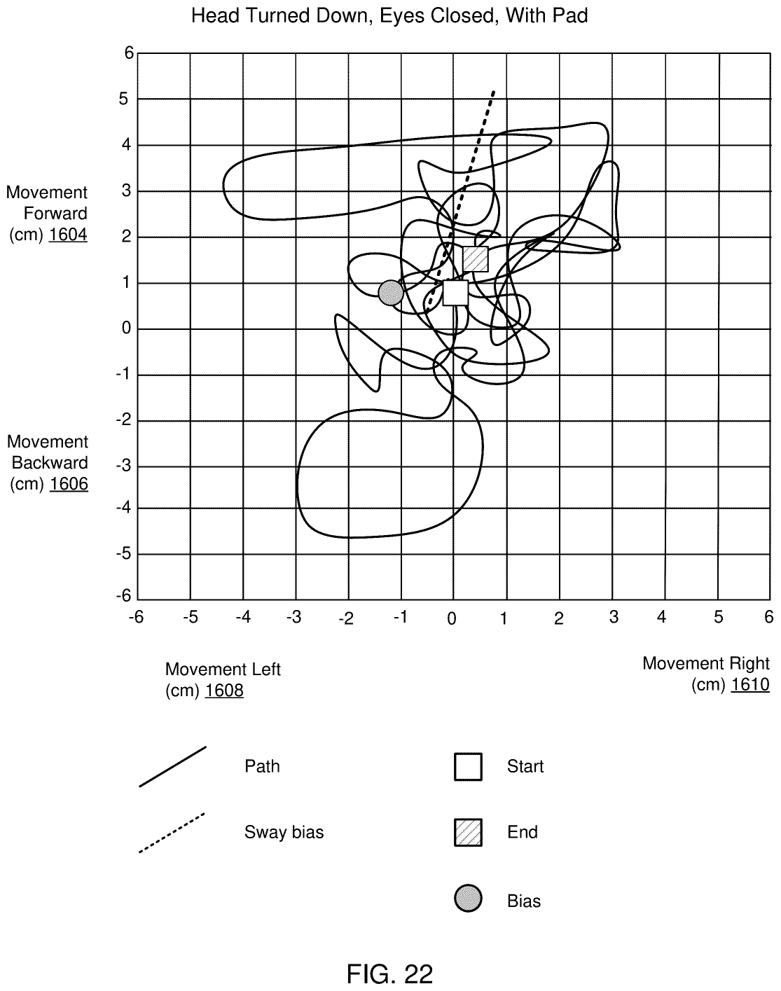

FIG. 22 is a plot of exemplary results of tracking posturography with a force platform 104 under the following assessment protocols: head turned downward, eyes closed, and with pad. The white block indicates the position of the force platform 104 in terms of forward/backward tilt and further in terms of rightward/leftward tilt, the dark line indicates the position of the force platform 104 in terms of forward/backward tilt and further in terms of rightward/leftward tilt over time, and the diagonal lined block indicates the final position of the force platform 104 in terms of forward/backward tilt and further in terms of rightward/leftward tilt.

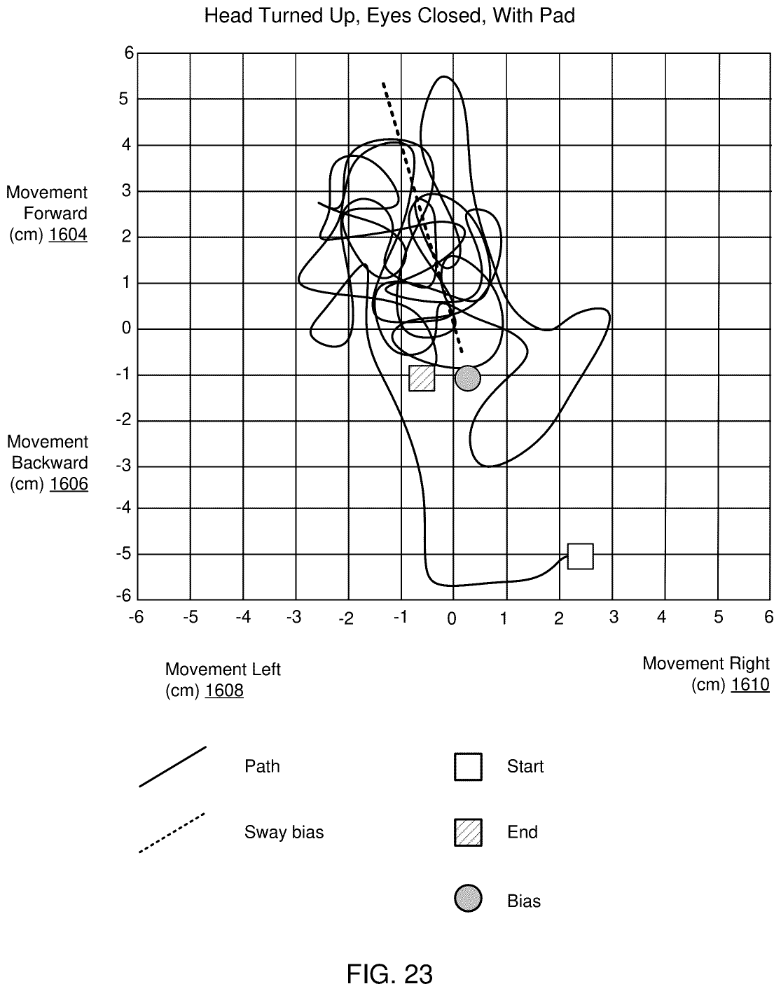

FIG. 23 is a plot of exemplary results of tracking posturography with a force platform 104 under the following assessment protocols: with turned upward, eyes closed, and with pad. The white block indicates the position of the force platform 104 in terms of forward/backward tilt and further in terms of rightward/leftward tilt, the dark line indicates the position of the force platform 104 in terms of forward/backward tilt and further in terms of rightward/leftward tilt over time, and the diagonal lined block indicates the final position of the force platform 104 in terms of forward/backward tilt and further in terms of rightward/leftward tilt.

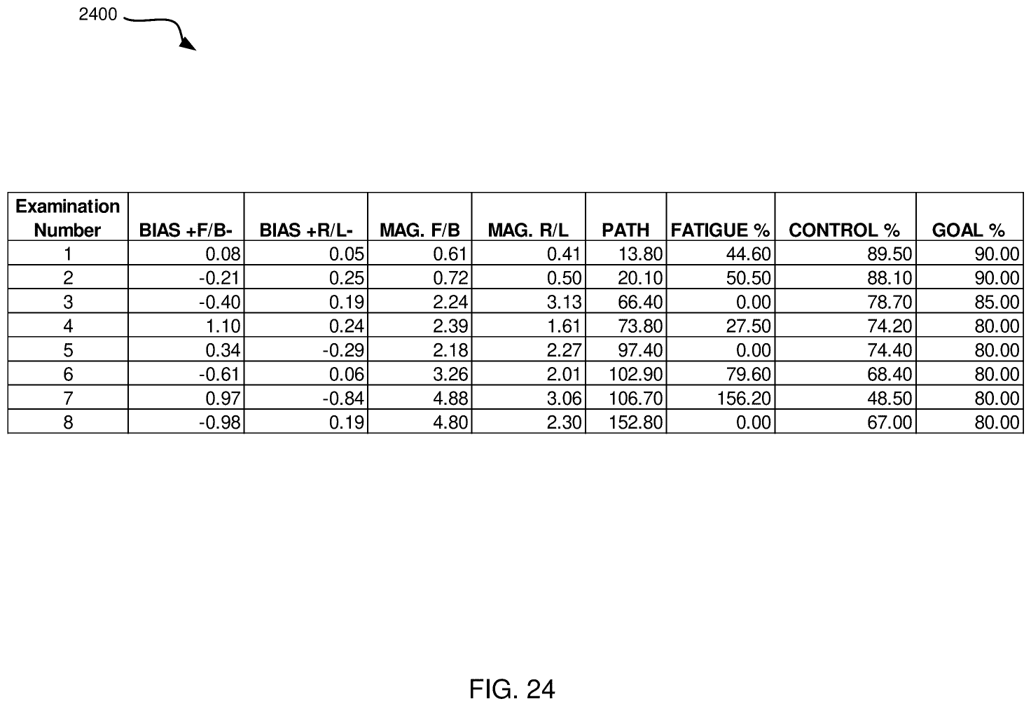

FIG. 24 is a chart illustrating example results 2400 of the tests illustrated in FIGS. 16-23. The example results 2400 may be rendered on a user interface and provided to a patient through the patient portal 110. The example results indicate the bias forward/backward, the bias right/left the magnitude forward/backward, the magnitude right/left, the path, the fatigue percentage, the control percentage, and the goal percentage for each test illustrated in FIGS. 16-23.

The bias represents a time-weighted average of a center of pressure signal output by the force platform 104 over a selected axis. The bias is calculated such that later time points (i.e., in a latter portion of the duration of the balance test duration) are weighted more heavily using quadratic time weighing. The bias may be calculated based upon Equation 1, below, wherein CoP represents the center of pressure signal, t represents time, and T represents the total duration of the balance test duration.

$$Bias = \sum {CoP}_{i} \frac{{( \frac{{t}_{i}}{T} )}^{2}}{\sum {( \frac{{t}_{i}}{T} )}^{2}} \qquad Equation 1$$

The magnitude is calculated based upon an absolute value of a 95th percentile of the center of pressure displacement along a selected axis. The magnitude reflects the extent of postural excursions. The magnitude may be calculated based on Equation 2, below.

$$Magnitude = \vert {P}_{95} ( {CoP}_{axis} ) \vert \qquad Equation 2$$

The fatigue is calculated based upon a weighted combination of a percent change in the center of pressure path length, an RMS sway amplitude, and a mean center of pressure velocity between the first and second halves of the balance test duration. The fatigue percentage may be calculated based upon Equation 3, below, wherein Δ represents a latter point in the balance test duration minus an earlier point in the balance test duration, and wherein w₁+w₂+w₃ is equal to one.

$$Fatigue \% = 100 \times ( {w}_{1} {Δ}_{path} ) + ( {w}_{2} {Δ}_{RMS} ) + ( {w}_{3} {Δ}_{velocity} ) \qquad Equation 3$$

The control is predicted based upon a gradient boosted regression model trained on four sway features, including a phase-plane parameter (LR), an LR range, a maximal LR distance, and a total two-dimensional sway length. The control is calculated based upon a series of data collected from numerous users and is trained based upon a linear regression model.

The goal represents a target value to define what is “good” for each of the tests and a target for the control score.

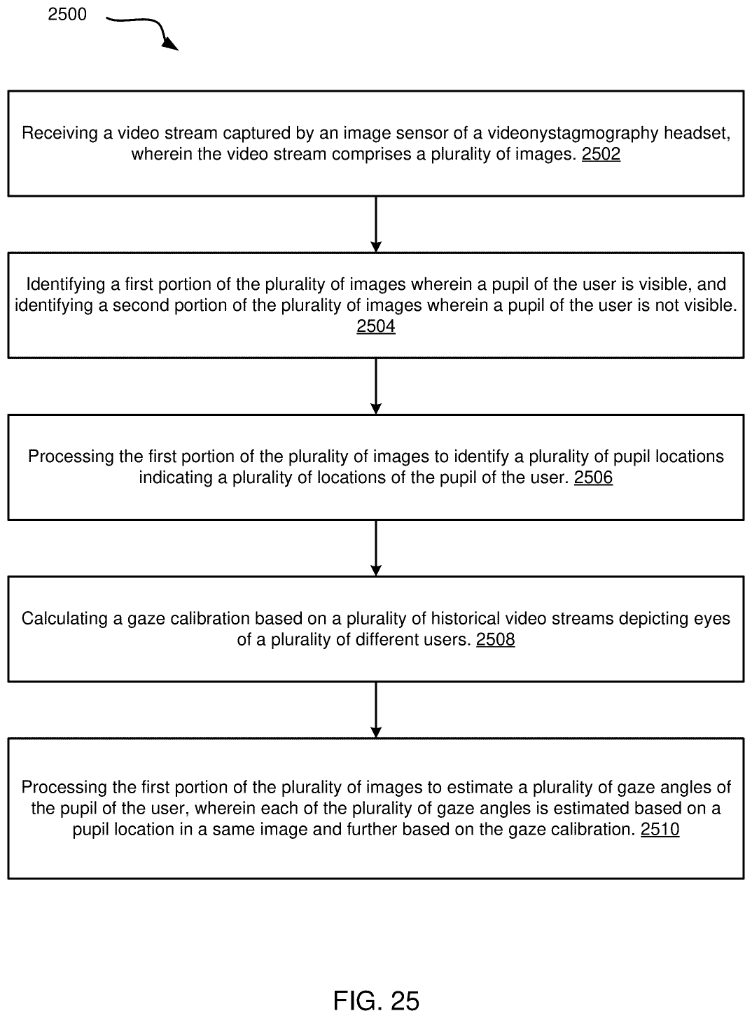

FIG. 25 is a schematic flow chart diagram of a method 2500 for assessing data output by a VNG system 102. The method 2500 is performed by a processor in communication with an image sensor of a VNG system 102.

The method 2500 includes receiving at 2502 a video stream captured by an image sensor of a videonystagmography (VNG) headset, wherein the video stream includes a plurality of images. The method 2500 includes identifying at 2504 a first portion of the plurality of images wherein a pupil of the user is visible, and identifying a second portion of the plurality of image wherein a pupil of the user is not visible. The method 2500 includes processing at 2506 the first portion of the plurality of images to identify a plurality of pupil locations indicating a plurality of locations of the pupil of the user. The method 2500 includes calculating at 2508 a gaze calibration based on a plurality of historical video streams depicting eyes of a plurality of different users. The method 2500 includes processing at 2510 the first portion of the plurality of images to estimate a plurality of gaze angles of the pupil of the user, wherein each of the plurality of gaze angles is estimated based on a pupil location in a same image and further based on the gaze calibration.

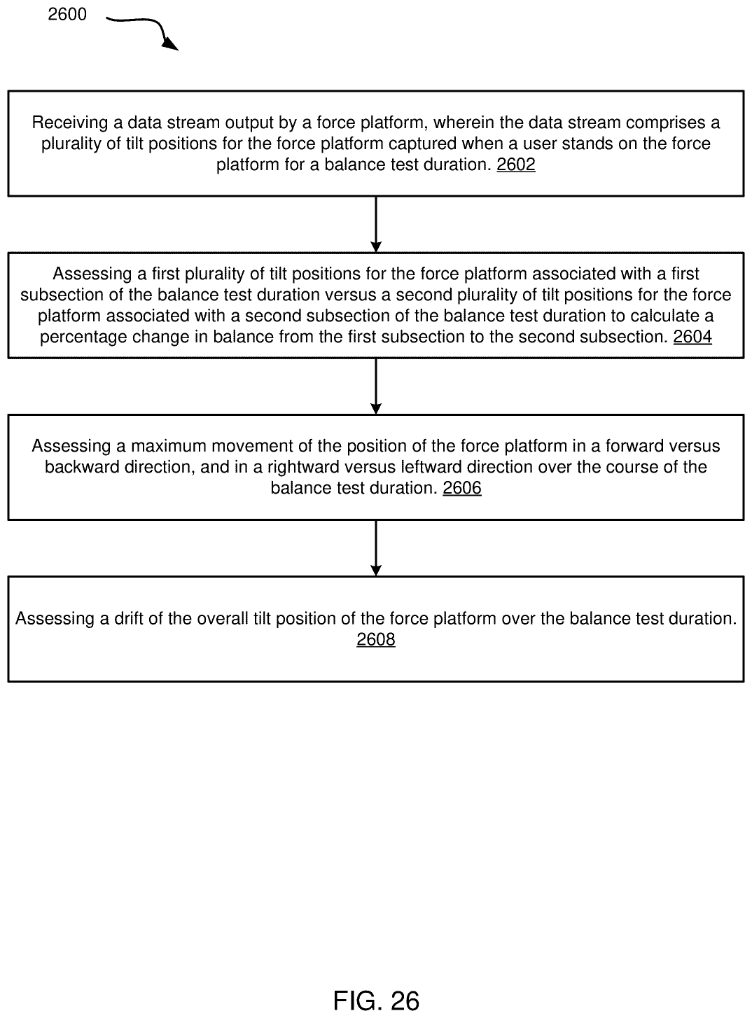

FIG. 26 is a schematic flow chart diagram of a method 2600 for assessing data output by a force platform 104. The method 2600 is performed by a processor that receives data output by the force platform 104.

The method 2600 includes receiving at 2602 a data stream output by a force platform, wherein the data stream includes a plurality of tilt positions for the force platform captured when a user stands on the force platform for a balance test duration. The method 2600 includes assessing at 2064 a first plurality of tilt positions for the force platform associated with a first subsection of the balance test duration versus a second plurality of tilt positions for the force platform associated with a second subsection of the balance test duration to calculate a percentage change in balance from the first subsection versus the second subsection of the balance test duration. The method 2600 includes assessing at 2606 a maximum movement of the position of the force platform in a forward versus backward direction, and in a rightward versus leftward direction over the course of the balance test duration. The method 2600 includes assessing at 2608 a drift of the overall tilt position of the force platform over the balance test duration.

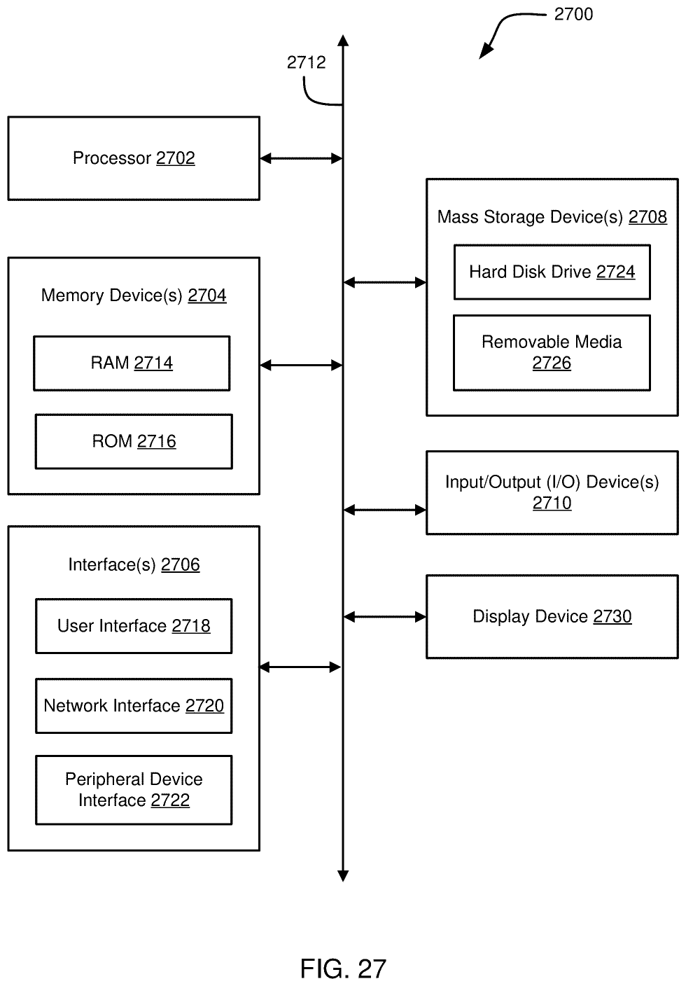

Referring now to FIG. 27, a block diagram of an example computing device 2700 is illustrated. Computing device 2700 may be used to perform various procedures, such as those discussed herein. Computing device 2700 can perform various monitoring functions as discussed herein, and can execute one or more application programs, such as the application programs or functionality described herein. Computing device 2700 can be any of a wide variety of computing devices, such as a desktop computer, in-dash computer, vehicle control system, a notebook computer, a server computer, a handheld computer, tablet computer and the like.

Computing device 2700 includes one or more processor(s) 2712, one or more memory device(s) 2704, one or more interface(s) 2706, one or more mass storage device(s) 2708, one or more Input/output (I/O) device(s) 2710, and a display device 2730 all of which are coupled to a bus 2712. Processor(s) 2712 include one or more processors or controllers that execute instructions stored in memory device(s) 2704 and/or mass storage device(s) 2708. Processor(s) 2712 may also include diverse types of computer-readable media, such as cache memory.

Memory device(s) 2704 include various computer-readable media, such as volatile memory (e.g., random access memory (RAM) 2714) and/or nonvolatile memory (e.g., read-only memory (ROM) 2716). Memory device(s) 2704 may also include rewritable ROM, such as Flash memory.

Mass storage device(s) 2708 include various computer readable media, such as magnetic tapes, magnetic disks, optical disks, solid-state memory (e.g., Flash memory), and so forth. As shown in FIG. 27, a particular mass storage device 2708 is a hard disk drive 2724. Various drives may also be included in mass storage device(s) 2708 to enable reading from and/or writing to the various computer readable media. Mass storage device(s) 2708 include removable media 2726 and/or non-removable media.

I/O device(s) 2710 include various devices that allow data and/or other information to be input to or retrieved from computing device 2700. Example I/O device(s) 2710 include cursor control devices, keyboards, keypads, microphones, monitors, touchscreen devices, or other display devices, speakers, printers, network interface cards, modems, and the like.

Display device 2730 includes any type of device capable of displaying information to one or more users of computing device 2700. Examples of display device 2730 include a monitor, display terminal, video projection device, and the like.

Interface(s) 2706 include various interfaces that allow computing device 2700 to interact with other systems, devices, or computing environments. Example interface(s) 2706 may include any number of different network interfaces 2720, such as interfaces to local area networks (LANs), wide area networks (WANs), wireless networks, and the Internet. Other interface(s) include user interface 2718 and peripheral device interface 2722. The interface(s) 2706 may also include one or more user interface elements 2718. The interface(s) 2706 may also include one or more peripheral interfaces such as interfaces for printers, pointing devices (mice, track pad, or any suitable user interface now known to those of ordinary skill in the field, or later discovered), keyboards, and the like.

Bus 2712 allows processor(s) 2712, memory device(s) 2704, interface(s) 2706, mass storage device(s) 2708, and I/O device(s) 2710 to communicate with one another, as well as other devices or components coupled to bus 2712. Bus 2712 represents one or more of several types of bus structures, such as a system bus, PCI bus, IEEE bus, USB bus, and so forth.

For purposes of illustration, programs and other executable program components are shown herein as discrete blocks, although it is understood that such programs and components may reside at various times in different storage components of computing device 1800 and are executed by processor(s) 2712. Alternatively, the systems and procedures described herein can be implemented in hardware, or a combination of hardware, software, and/or firmware. For example, one or more application specific integrated circuits (ASICs) can be programmed to conduct one or more of the systems and procedures described herein. As used herein, the terms “module” or “component” are intended to convey the implementation apparatus for accomplishing a process, such as by hardware, or a combination of hardware, software, and/or firmware, for the purposes of performing all or parts of operations disclosed herein. The terms “module” or “component” are intended to convey independent in how the modules, components, or their functionality or hardware may be implemented in different embodiments.

Various techniques, or certain aspects or portions thereof, may take the form of program code (i.e., instructions) embodied in tangible media, such as floppy diskettes, CD-ROMs, hard drives, a non-transitory computer readable storage medium, or any other machine-readable storage medium wherein, when the program code is loaded into and executed by a machine, such as a computer, the machine becomes an apparatus for practicing the various techniques. In the case of program code execution on programmable computers, the computing device may include a processor, a storage medium readable by the processor (including volatile and non-volatile memory and/or storage elements), at least one input device, and at least one output device. The volatile and non-volatile memory and/or storage elements may be a RAM, an EPROM, a flash drive, an optical drive, a magnetic hard drive, or another medium for storing electronic data. One or more programs that may implement or utilize the various techniques described herein may use an application programming interface (API), reusable controls, and the like. Such programs may be implemented in a high-level procedural, functional, object-oriented programming language to communicate with a computer system. However, the program(s) may be implemented in assembly or machine language, if desired. In any case, the language may be a compiled or interpreted language, and combined with hardware implementations.

It should be understood that many of the functional units described in this specification may be implemented as one or more components or modules, which are terms used to emphasize their implementation independence more particularly. For example, a component or module may be implemented as a hardware circuit comprising custom very large-scale integration (VLSI) circuits or gate arrays, off-the-shelf semiconductors such as logic chips, transistors, or other discrete components. A component may also be implemented in programmable hardware devices such as field programmable gate arrays, programmable array logic, programmable logic devices, and the like.

Components may also be implemented in software for execution by diverse types of processors. An identified component of executable code may, for instance, comprise one or more physical or logical blocks of computer instructions, which may, for instance, be organized as an object, a procedure, or a function. Nevertheless, the executables of an identified component need not be physically located together but may comprise disparate instructions stored in separate locations that, when joined logically together, comprise the component and achieve the stated purpose for the component.

Indeed, a component of executable code may be a single instruction, or many instructions, and may even be distributed over several different code segments, among different programs, and across several memory devices. Similarly, operational data may be identified and illustrated herein within components and may be embodied in any suitable form and organized within any suitable type of data structure. The operational data may be collected as a single data set or may be distributed over separate locations including over different storage devices, and may exist, at least partially, merely as electronic signals on a system or network. The components may be passive or active, including agents operable to perform desired functions.

### EXAMPLES

The following examples pertain to further embodiments.

Example 1 is a system. The system includes a videonystagmography headset comprising an image sensor, wherein the image sensor outputs a video stream comprising a plurality of images of eyes of a user captured when the user wears the videonystagmography headset for a gaze test duration. The system includes a force platform comprising a load cell, wherein the force platform outputs a data stream indicating a plurality of tilt positions of the force platform captured when the user stands on the force platform for a balance test duration. The system includes a computer processor that executes instructions stored in non-transitory computer readable storage medium. The instructions include processing the video stream to estimate a plurality of gaze angles of the eyes of the user over the gaze test duration. The instructions includes processing the data stream to categorize a balance of the user based upon movement of the force platform over the balance test duration.

Example 2 is a system as in Example 1, wherein the instructions further comprise: receiving the video stream comprising the plurality of images; identifying a first portion of the plurality of images wherein a pupil of the user is visible; identifying a second portion of the plurality of images wherein a pupil of the user is not visible; processing the first portion of the plurality of images to identify a plurality of pupil locations indicating a plurality of locations of the pupil of the user; processing the first portion of the plurality of images to estimate a plurality of gaze angles of the pupil of the user; wherein each of the plurality of gaze angles is estimated based on a pupil location in a same image; wherein the plurality of gaze angles is further estimated based on a gaze calibration; and wherein the gaze calibration is determined based on a plurality of historical video streams depicting eyes of a plurality of different users.

Example 3 is a system as in any of Examples 1-2, wherein the load cell measures an equivalent force applied to a surface of the force platform at a point of application, and wherein the force platform outputs data pertaining to a center of pressure on the force platform, and wherein the force platform outputs data pertaining to a vertical moment of force applied to the force platform.

Example 4 is a system as in any of Examples 1-3, wherein processing the data stream to categorize the balance of the user comprises calculating a fatigue of the user over the balance test duration, wherein calculating the fatigue of the user comprises: identifying an initial average position of the force platform when the user stands on the force platform for an initial subsection of the balance test duration; identifying a final average position of the force platform when the user stands on the force platform for a final subsection of the balance test duration; and comparing the initial average position against the final average position to calculate a percentage change of the position of the force platform during the initial subsection of the balance test duration versus the final subsection of the balance test duration.

Example 5 is a system as in any of Examples 1-4, wherein processing the data stream to categorize the balance of the user comprises calculating a path of the force platform over the balance test duration, wherein the path of the force platform indicates an average sway distance for the force platform.

Example 6 is a system as in any of Examples 1-5, wherein processing the data stream to categorize the balance of the user comprises calculating a magnitude of the force platform over the balance test duration, wherein the magnitude of the force platform comprises: a forward/backward magnitude indicating a maximum sway of the force platform forward or backward over the balance test duration; and a right/left magnitude indicating a maximum sway of the force platform rightward or leftward over the balance test duration.

Example 7 is a system as in any of Examples 1-6, wherein processing the data stream to categorize the balance of the user comprises calculating a bias, wherein the bias indicates which direction the force platform moved most often during the balance test duration.

Example 8 is a system as in any of Examples 1-7, wherein processing the data stream to categorize the balance of the user comprises one of: categorizing the user as having stable balance if the movement of the force platform over the balance test duration remained within a stable threshold; categorizing the user as having an unstable balance if the movement of the force platform over the balance test duration was outside the stable threshold.

Example 9 is a system as in any of Examples 1-8, wherein the videonystagmography headset further comprise a stimuli projector, and wherein the stimuli projector emits a plurality of visual stimuli to the eyes of the user; and wherein the stimuli projector of the videonystagmography headset projects at least a portion of the plurality of visual stimuli to the eyes of the user concurrently with the image sensor of the videonystagmography headset capturing at least one image of the plurality of images of the eyes of the user.

Example 10 is a system as in any of Examples 1-9, wherein the plurality of images of the eyes of the user depict each of a left eye of the user and a right eye of the user; and wherein the instructions executed by the processor further comprise splitting each of the plurality of images into a left-eye image and a right image to generate a plurality of left-eye images a plurality of right-eye images.

Example 11 is a system as in any of Examples 1-10, wherein the instructions executed by the processor further comprise associating metadata with each of the plurality of left-eye images and each of the plurality of right-eye images, and wherein the metadata comprises a total frame count of the video stream and a frame rate of the video stream; and wherein the metadata further comprises a stimuli location emitted by a stimuli projector of the videonystagmography headset, wherein the stimuli location corresponds in time with a capture time of an associated image such that the stimuli location indicates a position of visual stimuli when the image sensor captured the associated image.

Example 12 is a system as in any of Examples 1-11, wherein the instructions executed by the processor are such that processing the first portion of the plurality of images to identify the plurality of pupil locations comprises: separately processing a first portion of the plurality of left-eye images and a first portion of the plurality of right-eye images; calculating a plurality of left-eye pupil locations based on the first portion of the plurality of left-eye images; and calculating a plurality of right-eye pupil locations based on the first portion of the plurality of right-eye images.

Example 13 is a system as in any of Examples 1-12, wherein the instructions executed by the processor are such that processing the first portion of the plurality of images to estimate the plurality of gaze angles of the pupil of the user comprises: separately processing the first portion of the plurality of left-eye images and the first portion of the plurality of right-eye images; calculating a plurality of left-eye gaze angles based on the first portion of the plurality of left-eye images; and calculating a plurality of right-eye gaze angles based on the first portion of the plurality of right-eye images.

Example 14 is a system as in any of Examples 1-13, wherein the instructions executed by the processor further include generating a plot illustrating eye movement of the user, wherein the plot comprises: a first axis depicting a passage of time; a second axis depicting the eye movement of the user measured in degrees; a first plot line depicting the plurality of left-eye gaze angles over time; and a second plot line depicting the plurality of right-eye gaze angles over time.

Example 15 is a system as in any of Examples 1-14, wherein the plot depicts vertical eye movement such that the second axis comprises: positive degrees indicating upward vertical eye movement; and negative degrees indicating downward vertical eye movement.

Example 16 is a system as in any of Examples 1-15, wherein the plot depicts horizontal eye movement such that the second axis comprises: positive degrees indicating rightward horizontal eye movement; and negative degrees indicating leftward horizontal eye movement.

Example 17 is a system as in any of Examples 1-16, wherein the instructions executed by the processor further comprise: synchronizing a plurality of left-eye gaze angles and a plurality of right-eye gaze angles based on capture time to generate a plurality of gaze angle pairs; and comparing each of the plurality of gaze angles pairs to identify whether the eyes of the user ever converge during the video stream; wherein the eyes of the user converge when a value of a horizontal difference between a left-eye gaze angle and a right-eye gaze angle exceed a convergence threshold.

Example 18 is a system as in any of Examples 1-17, wherein the instructions executed by the processor further comprise: assessing the plurality of left-eye gaze angles to determine whether the left eye of the user ever engages in a rapid eye movement during the video stream; and assessing the plurality of right-eye gaze angles to determine whether the right eye of the user ever engages in the rapid eye movement during the video stream; wherein the rapid eye movement occurs when one or more of the left eye or the right eye of the user moves at a velocity exceeding a threshold, and further when the left eye or the right eye of the user moves in a direction different from a direction of movement of the visual stimuli at a same point in time.

Example 19 is a system as in any of Examples 1-18, wherein the instructions executed by the processor are such that processing the first portion of the plurality of images to identify the plurality of pupil locations comprises: for each image of the first portion of the plurality of images, identifying one or more pixels depicting a center point of the pupil of the user; and for each image of the first portion of the plurality of images, calculating an ellipse fit to a geometry of the pupil of the user.

Example 20 is a system as in any of Examples 1-19, wherein the instructions executed by the processor further comprise, for each image of the first portion of the plurality of images, normalizing pixel values for the one or more pixels depicting the center point of the pupil of the user; wherein normalizing the pixel values comprises normalizing based on an estimated eye gaze zero-angle location; and wherein the estimated eye gaze zero-angle location is calculated based on the historical video streams depicting the eyes of the plurality of different users.

Example 21 is a system as in any of Examples 1-20, wherein the instructions executed by the processor further comprise classifying each image of the second portion of the plurality of images as depicting a blink by the user.

Example 22 is a system as in any of Examples 1-21, wherein the instructions executed by the processor further comprise: identifying two or more consecutive images of the second portion of the plurality of images, wherein the two or more consecutive images were captured consecutively by the image sensor of the videonystagmography system; classifying the two or more consecutive images as applying to a first blink by the user; calculating a quantity of the two or more consecutive images; and calculating a duration of the first blink by the user based on the quantity of the two or more consecutive images and further based on a frame rate of the video stream.

Example 23 is a system as in any of Examples 1-22, wherein the instructions executed by the processor further comprise: calculating a number of blinks performed by the user over the video stream, wherein each of the blinks is separated by one or more images wherein the pupil of the user is visible; and calculating a blink rate for the user indicating a number of blinks per time period.

Reference throughout this specification to “an example” means that a particular feature, structure, or characteristic described in connection with the example is included in at least one embodiment of the present disclosure. Thus, appearances of the phrase “in an example” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present disclosure may be referred to herein along with alternatives for the various components thereof. It is understood that such embodiments, examples, and alternatives are not to be construed as de facto equivalents of one another but are to be considered as separate and autonomous representations of the present disclosure.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive.

Those having skill in the art will appreciate that many changes may be made to the details of the above-described embodiments without departing from the underlying principles of the disclosure.

What is claimed is:

1. A system comprising:
a videonystagmography headset comprising an image sensor, wherein the image sensor outputs a video stream comprising a plurality of images of eyes of a user captured when the user wears the videonystagmography headset for a gaze test duration;
a force platform comprising a load cell, wherein the force platform outputs a data stream indicating a plurality of tilt positions of the force platform captured when the user stands on the force platform for a balance test duration; and
a computer processor that executes instructions stored in non-transitory computer readable storage medium, the instructions comprising:
processing the video stream to estimate a plurality of gaze angles of the eyes of the user over the gaze test duration; and
processing the data stream to categorize a balance of the user based upon tilt movement of the force platform over the balance test duration.

2. The system of claim 1, wherein the instructions further comprise:
receiving the video stream comprising the plurality of images;
identifying a first portion of the plurality of images wherein a pupil of the user is visible;
identifying a second portion of the plurality of images wherein a pupil of the user is not visible;
processing the first portion of the plurality of images to identify a plurality of pupil locations indicating a plurality of locations of the pupil of the user;
processing the first portion of the plurality of images to estimate a plurality of gaze angles of the pupil of the user;
wherein each of the plurality of gaze angles is estimated based on a pupil location in a same image;
wherein the plurality of gaze angles is further estimated based on a gaze calibration; and
wherein the gaze calibration is determined based on a plurality of historical video streams depicting eyes of a plurality of different users.

3. The system of claim 1, wherein processing the data stream to categorize the balance of the user comprises calculating a fatigue of the user over the balance test duration, wherein calculating the fatigue of the user comprises:
identifying an initial average position of the force platform when the user stands on the force platform for an initial subsection of the balance test duration;
identifying a final average position of the force platform when the user stands on the force platform for a final subsection of the balance test duration; and
comparing the initial average position against the final average position to calculate a percentage change of the position of the force platform during the initial subsection of the balance test duration versus the final subsection of the balance test duration.

4. The system of claim 1, wherein processing the data stream to categorize the balance of the user comprises calculating a path of the force platform over the balance test duration, wherein the path of the force platform indicates an average sway distance for the force platform.

5. The system of claim 1, wherein processing the data stream to categorize the balance of the user comprises calculating a balance magnitude for the user, wherein the balance magnitude comprises:
a forward/backward magnitude indicating a maximum sway of the force platform forward or backward over the balance test duration; and
a right/left magnitude indicating a maximum sway of the force platform rightward or leftward over the balance test duration.

6. The system of claim 1, wherein processing the data stream to categorize the balance of the user comprises calculating a bias, wherein the bias indicates which direction the force platform moved most often during the balance test duration.

7. The system of claim 1, wherein processing the data stream to categorize the balance of the user comprises one of:
categorizing the user as having stable balance if the movement of the force platform over the balance test duration remains within a stable threshold; or
categorizing the user as having an unstable balance if the movement of the force platform over the balance test duration was outside the stable threshold.

8. The system of claim 1, wherein the videonystagmography headset further comprises a stimuli projector, and wherein the stimuli projector emits a plurality of visual stimuli to the eyes of the user; and
wherein the stimuli projector of the videonystagmography headset projects at least a portion of the plurality of visual stimuli to the eyes of the user concurrently with the image sensor of the videonystagmography headset capturing at least one image of the plurality of images of the eyes of the user.

9. The system of claim 1, wherein the plurality of images of the eyes of the user depict each of a left eye of the user and a right eye of the user; and
wherein the instructions executed by the processor further comprise splitting each of the plurality of images into a left-eye image and a right image to generate a plurality of left-eye images a plurality of right-eye images.

10. The system of claim 9, wherein the instructions executed by the processor further comprise associating metadata with each of the plurality of left-eye images and each of the plurality of right-eye images, and wherein the metadata comprises a total frame count of the video stream and a frame rate of the video stream; and
wherein the metadata further comprises a stimuli location emitted by a stimuli projector of the videonystagmography headset, wherein the stimuli location corresponds in time with a capture time of an associated image such that the stimuli location indicates a position of visual stimuli when the image sensor captured the associated image.

11. The system of claim 2, wherein the instructions executed by the processor are such that processing the first portion of the plurality of images to identify the plurality of pupil locations comprises:
separately processing a first portion of the plurality of left-eye images and a first portion of the plurality of right-eye images;
calculating a plurality of left-eye pupil locations based on the first portion of the plurality of left-eye images; and
calculating a plurality of right-eye pupil locations based on the first portion of the plurality of right-eye images.

12. The system of claim 2, wherein the instructions executed by the processor are such that processing the first portion of the plurality of images to estimate the plurality of gaze angles of the pupil of the user comprises:
separately processing the first portion of the plurality of left-eye images and the first portion of the plurality of right-eye images;
calculating a plurality of left-eye gaze angles based on the first portion of the plurality of left-eye images; and
calculating a plurality of right-eye gaze angles based on the first portion of the plurality of right-eye images.

13. The system of claim 1, wherein the instructions executed by the processor further include generating a plot illustrating eye movement of the user, wherein the plot comprises:
a first axis depicting a passage of time;
a second axis depicting the eye movement of the user measured in degrees;
a first plot line depicting the plurality of left-eye gaze angles over time; and
a second plot line depicting the plurality of right-eye gaze angles over time.

14. The system of claim 13, wherein the plot depicts vertical eye movement such that the second axis comprises: positive degrees indicating upward vertical eye movement; and negative degrees indicating downward vertical eye movement; and
wherein the plot depicts horizontal eye movement such that the second axis comprises: positive degrees indicating rightward horizontal eye movement; and negative degrees indicating leftward horizontal eye movement.

15. The system of claim 2, wherein the instructions executed by the processor further comprise:
synchronizing a plurality of left-eye gaze angles and a plurality of right-eye gaze angles based on capture time to generate a plurality of gaze angle pairs; and
comparing each of the plurality of gaze angles pairs to identify whether the eyes of the user ever converge during the video stream;
wherein the eyes of the user converge when a value of a horizontal difference between a left-eye gaze angle and a right-eye gaze angle exceed a convergence threshold.

16. The system of claim 15, wherein the instructions executed by the processor further comprise:
assessing the plurality of left-eye gaze angles to determine whether the left eye of the user ever engages in a rapid eye movement during the video stream; and
assessing the plurality of right-eye gaze angles to determine whether the right eye of the user ever engages in the rapid eye movement during the video stream;
wherein the rapid eye movement occurs when one or more of the left eye or the right eye of the user moves at a velocity exceeding a threshold, and further when the left eye or the right eye of the user moves in a direction different from a direction of movement of the visual stimuli at a same point in time.

17. The system of claim 2, wherein the instructions executed by the processor are such that processing the first portion of the plurality of images to identify the plurality of pupil locations comprises:
for each image of the first portion of the plurality of images, identifying one or more pixels depicting a center point of the pupil of the user; and
for each image of the first portion of the plurality of images, calculating an ellipse fit to a geometry of the pupil of the user.

18. The system of claim 2, wherein the instructions executed by the processor further comprise, for each image of the first portion of the plurality of images, normalizing pixel values for the one or more pixels depicting the center point of the pupil of the user;
wherein normalizing the pixel values comprises normalizing based on an estimated eye gaze zero-angle location; and
wherein the estimated eye gaze zero-angle location is calculated based on the historical video streams depicting the eyes of the plurality of different users.

19. The system of claim 2, wherein the instructions executed by the processor further comprise:
identifying two or more consecutive images of the second portion of the plurality of images, wherein the two or more consecutive images were captured consecutively by the image sensor of the videonystagmography system;
classifying the two or more consecutive images as applying to a first blink by the user;
calculating a quantity of the two or more consecutive images; and
calculating a duration of the first blink by the user based on the quantity of the two or more consecutive images and further based on a frame rate of the video stream.

20. The system of claim 1, wherein the instructions executed by the processor further comprise:
calculating a number of blinks performed by the user over the video stream, wherein each of the blinks is separated by one or more images wherein the pupil of the user is visible; and
calculating a blink rate for the user indicating a number of blinks per time period.
